(19) United States
(12) Patent Application Publication
(10) Pub. No.: US 2018/0312855 A1
(43) Pub. Date: Nov. 1, 2018

(54) ALGAL PROMOTERS

(71) Applicant: B. G. NEGEV TECHNOLOGIES AND APPLICATIONS LTD., AT BEN-GURION UNIVERSITY, Beer Sheva (IL)

(72) Inventors: Sammy BOUSSIBA, Omer (IL); Inna KHOZIN-GOLDBERG, Midreshet Ben Gurion (IL); Zachor SHEMESH, Midreshet Ben Gurion (IL); Stefan LEU, Midreshet Ben Gurion (IL); Aliza ZARKA, Beer Sheva (IL)

(21) Appl. No.: 15/739,762

(22) PCT Filed: Jun. 23, 2016
(86) PCT No.: PCT/IL2016/050672

§ 371 Date: Dec. 25, 2017

Related U.S. Application Data
(60) Provisional application No. 62/183,762, filed on Jun. 24, 2015.

(51) Int. Cl. C12N 15/82; C12N 15/67; C07K 14/405; C12N 15/113; C12P 7/64

(52) U.S. Cl. CPC C12N 15/8216; C12N 15/67; C12N 15/743; C12N 15/113; C12P 7/6472; C07K 14/405

(57) ABSTRACT

The present invention provides a nucleic acid molecule comprising: DGAT1 promoter or an active fragment thereof, the ammonium transporter promoter or an active fragment thereof, or the putative purine permease promoter or an active fragment thereof; coupled to a gene. The invention further provides a vector including the nucleic acid molecule and cells harboring the nucleic acid molecule.

### FIELD OF THE INVENTION

The present invention provides promoters derived from alga or microalga that are capable, inter-alia, in inducing the expression of genes of interest under specific physiological conditions such as nitrogen starvation and/or are constitutively active.

### BACKGROUND OF THE INVENTION

Microalgae are evolutionary diverse unicellular, eukaryotic organisms. Microalgae use sunlight to produce biomass and oxygen from CO₂, water and nutrients. Microalgae can be cultivated in marginal or saline water on unproductive dryland and thus do not compete with agriculture for arable land or fresh water. This makes microalgae a promising resource for highly sustainable production of various biomaterials, biochemicals or biofuels (Benemann et al. 1987; Leu & Boussiba 2014; Wijffels & Barbosa 2010) especially in the large available sun rich dryland areas where other agricultural activities are restricted. Microalgae have high areal biomass productivity and can accumulate significant concentrations of high value products, or over 50% of dry weight as oil in the form of triacylglycerol (TAG) under adequate conditions (Pulz & Gross 2004; Priyadarshani & Rath 2012; Rodolfi et al. 2009; Axelsson et al. 2012). In contrast to higher plants, over 90% of the algal biomass can be valorized into feed, food, energy or high value chemicals, so that application of biorefinery approaches can yield a wide range of useful products.

Recent developments in genetic engineering of various microalgae species among them the diatom Phaeodactylum tricornutum, promise rapid progress in strain improvements by metabolic engineering. However, the absence of suitable stage specific promoters able to drive gene expression under desired condition such as nitrogen starvation conditions is still a limiting factor in the production of fatty acids such as long-chain polyunsaturated fatty acids (LC-PUFAs), study of algal cell biology and genetic engineering for enhanced biofuels or oil production. This is especially grave because promoter sequences within the various microalgae kingdoms are highly variable and no conserved motifs or regulatory signals within microalgal promoter sequences have been described.

P. tricornutum is a well-studied diatom of significant economic potential, currently used primarily in aquaculture (Raja et al. 2008) and as a model organism. Its genomic DNA sequence was determined, and specific molecular tools, such as transformation and RNA interference, have been developed for P. tricornutum (Zaslayskaia et al. 2000; De Riso et al. 2009) Moreover, a large P. tricornutum EST library (Maheswari et al. 2010) has been created, and a wide range of gene expression data under varying conditions have been assembled by quantitative transcriptomics under different growth conditions, including nitrogen starvation (Valenzuela et al. 2012). Expression and production of foreign proteins, such as monoclonal human antibodies in Phaeodactylum tricornutum have been achieved (Hempel et al., 2011; Vanier et al., 2015).

Microalgae are one of the richest sources of long-chain polyunsaturated fatty acids (LC-PUFAs). The green freshwater microalga Parietochloris incisa (Trebouxiophyceae) is the only microalga able to accumulate extraordinary high amounts of LC-PUFA arachidonic acid (ARA)-rich triacylglycerols (TAG). When P. incisa is cultivated under nitrogen starvation, the fatty acid (FA) content of the alga is over 35% of dry weight; ARA constitutes about 60% of total FAs, and over 90% of cell ARA is deposited in TAG, making it the richest green dietary source of ARA. LC-PUFAs include the ω3-fatty acids, eicosapentaenoic acid (EPA, 20:5ω3), docosahexaenoic acid (DHA, 22:6ω3), ω6-fatty acid, arachidonic acid (ARA, 20:4ω6) and dihomo-γ-linolenic acid (DGLA, 20:3ω6). LC-PUFA are major components of membrane phospholipids of the retina, brain and testis and are predominant in the human brain and breast milk (specifically ARA and DHA). ARA is necessary for normal fetal growth and for cognitive development in infants and is also the primary substrate in eicosanoids biosynthesis, which regulates many physiological processes such as homeostasis, reproduction, immune and inflammatory responses.

Production and accumulation of TAG and LDs is a stress induced process of great importance in biotechnology of microalgae, due to rising interest in oil production by microalgae for biofuels production. In order to engineer strains more efficiently for accumulation of TAG, promoters active under nitrogen stress must be applied. Furthermore, P. tricornutum is also a suitable tool to overexpress high value proteins for medical or biotech applications. Therefore, identification and application of novel promoters ensuring highest possible constitutive or stress-inducible gene expression must be therefore identified.

Investigation and modulation of TAG biogenesis in algae by genetic engineering, requires the application of promoters active also under nitrogen starvation.

Although a number of novel promoters have been described and tested in P. tricornutum, such as viral promoters (Sakaue et al. 2008b) and iron-responsive promoters (Yoshinaga et al. 2014), none of them have been found suitable for use under nitrogen starvation conditions.

There remains an unmet need for the development of cells, in particular microalgal cells which are capable of producing LC-PUFA in large scale systems and which are resistant to herbicides, using methods which do not classify the organism as genetically modified.

### SUMMARY OF THE INVENTION

According to one aspect, the present invention provides a nucleic acid molecule comprising DGAT1 promoter or an active fragment thereof coupled to a gene, wherein the gene is not controlled endogenically by the DGAT1 promoter.

According to one embodiment the present invention further provides, a nucleic acid molecule comprising ammonium transporter promoter or an active fragment thereof coupled to a gene, wherein the gene is not controlled endogenically by the ammonium transporter promoter.

According to one embodiment the present invention further provides, a nucleic acid molecule comprising putative purine permease promoter or an active fragment thereof coupled to a gene, wherein the gene is not controlled endogenically by the putative purine permease promoter.

According to one embodiment the present invention further provides a method for enhancing the production of a protein, comprising the steps of: (a) preparing a composite nucleic acid molecule of a promoter of the invention or an active fragment thereof; and (b) transforming a cell with the nucleic acid molecule, thereby enhancing the production of a protein in a cell,

### BRIEF DESCRIPTION OF THE DRAWINGS

FIG. 1. is a schematic representation of Constructs created for studying RNA expression accumulation of the EGFP-PM fusion protein during N-starvation, driven by different promoters.

FIG. 2. is a micrograph showing accumulation of EGFP in cells transformed with EGFP-PM constructs during nitrogen starvation. A) fcp-A promoter at time 0, of starvation; B) fcp-A promoter at day 7 of nitrogen starvation; C) Purine permease promoter at time 0, of nitrogen starvation; D) Purine permease promoter at day 7 of nitrogen starvation as visualized by fluorescence microscopy in a representative transformant strains; only the overlay of the light microscopy with the GFP-fluorescence is shown;

FIG. 3. is a gel micrograph showing quantification of the GFP-PA fusion protein in recombinant strains at time 0, and after one, 3 and 7 days of nitrogen starvation, as determined by Western Blot analysis using anti-GFP antibody.

FIG. 4. is a bar graph showing mRNA levels for the GFP-PM constructs driven by the different promoters indicated as determined by qRT-PCR, after 0, 24, 48 hours and seven days of nitrogen starvation.

FIG. 5. provides a schematic representation (A) and a micrograph (B): GFP-HOGP localizes to lipid droplets induced by palmitoleic acid administration to nutrient-replete culture of P. tricornutum. Fluorescent protein was observed upon expression using the fcpA promoter and the DGAT1 promoter (A) Vector constructs for expression of the -HOGP fusion protein in P. tricornutum. Top: under control of the fcpA promoter, bottom: under control of the promoter of P. tricornutum DGA T/(B) In vivo localization of EGFP-HOGP expressed in P. tricornutum cells supplemented with palmitoleic acid *Rec: Twenty four hours following 16:1 addition, cells were pelleted and recovered in fresh medium. LM: light microscopy; PAF: photosynthetic apparatus fluorescence; GFP: GFP-HOGP fusion protein fluorescence; Merge: overlay of LM and GFP.

FIG. 6. provides a gel micrograph (A) and a bar graph (B): Quantification of the eGFP-HOGP fusion protein and expression of the HOGP mRNA in recombinant strains and wild type in the course of LDs formation upon feeding with palmitoleic acid and recovery following medium replacement (A) Western blot analysis on P tricornutum transformant lines expressing GFP-HOGP fusion proteins. (B) Expression levels of the HOGP mRNA under control of the fcp-A promoter (white bars) or DGAT1 promoter (black bars) during LD formation determined by qPCR.

FIG. 7. is a micrograph showing in vivo localization of GFP-HOGP expressed under nitrogen starvation in P. tricornutum. Confocal micrographs of a fcpA-GFP-HOGP transformant strain, and a DGAT1-GFP-HOGP transformant strain under nitrogen starvation conditions. Only the latter reveals a clear GFP signal localized to the LDs under nitrogen starvation.

FIG. 8. provides a gel micrograph and a bar graph showing quantification of the eGFP-HOGP fusion protein by Western blotting and expression level of the HOGP mRNA in recombinant strains and wild type in the course of LDs formation under nitrogen starvation(A) EHOGP-GFP accumulation in P. tricornutum transformant lines exposed to nitrogen starvation for 0, 3 and 7 days upon expression under the control of the fcpA promoter (upper lane), and under control of the P. tricornutum DGAT1 promoter in two independent transformant lines C3 and C18. (B) Expression levels of EGFP-HOGP mRNA at the onset (0) and after 3, and 7 days of nitrogen starvation under control of the fcpA promoter or under control of the DGAT1 promoter in two transgene lines, C3 and C18. The wild type strain was used as a negative control.

FIG. 9. provides bar graphs showing HOGP overexpression impacts total fatty acid content of biomass and volumetric concentration. (A) Total fatty acids content (% of dry weight) in wild type, and in two independent transformant lines C3 and C18, transformed with the DGAT1::GFP-HOGP construct, induced by nitrogen starvation. a, b and c indicate a significant difference between wild type and transformants (C3 and C18) according to the Tukey post hoc test with p<0.0001. Results are shown as the mean±standard deviation (n=6). (B) Accumulation of TAG in wild type and in transformants C3 and C18 after 7 days of nitrogen starvation. The differences observed between wild type and transformants (C3 and C18) are significant according to the Tukey post hoc test with p<0.0001. Results are shown as the mean±standard deviation (n=9). (C) Fatty acid composition of TAG in wild type and in transformants C3 and C18 after 7 days of nitrogen starvation. Results are shown as the mean±standard deviation (n=3).

FIG. 10. is a bar graph showing total fatty acids content in wild type, and in three independent transformant lines B8 C2 and G4, transformed with the AT::mKate2-HOGP construct, after 7 days of nitrogen starvation. *** A, B and C indicate a significant difference between treatments according to the Tukey post hoc test with p<0.0001. Results are expressed as the mean±standard deviation (n=6).

FIG. 11. is a bar graph showing the accumulation of TAG in wild type in three independent transformant lines B8, C2 and G4, transformed with the AT::mKate2-HOGP construct after 7 days of nitrogen starvation. The differences observed between wild type and the three independent transformant lines are significant with p<0.0001 ***. Results are expressed as the mean±standard deviation (wild type n=12; Transformant lines n=9).

FIG. 12. is a micrograph showing on-colony PCR of P. tricornutum transformed with the plasmids. The chosen colonies and WT as a control were grown for 7 days as follows: all colonies grew under nitrogen starvation (−N) to induce the expression of DGAT1 and AT promoters and TAG accumulation; in addition the AT promoter transformed colonies were grown in full nitrogen medium (+N) since the AT is expected to be expressed also under these conditions.

FIG. 13. is a micrograph showing P. tricornutum WT and D1 transformant after 5 days of growth in full RSE medium (+N) and after 5 days of nitrogen starvation (−N). Lipid droplets were formed under nitrogen starvation in both strains while in D1 a small amount of lipid bodies formed also under N-replete growth.

FIG. 14. is a bar graph showing growth of P. tricornutum in complete (blue lines) and nitrogen deplete (red lines) RSE medium. Presented the growth of WT and selected transformants harboring the constructs: AT promoter+PtDGAT1 (D1, E1), DGAT1 promoter+PtDGAT1 (D6). Values represent the DW averages of the WT and the transformants measured in days 0, 3 and 7.

FIG. 15. is a micrograph showing total lipids separation by TLC from P. tricornutum transformants and WT harvested after 7 days of (A) growth in full RSE medium (nitrogen-replete) and (B) under nitrogen starvation. Lipid extracts were separated and individual spots were visualized by charring. St: neutral lipid standards. Transgene algae transformed with the construct AT promoter-DGAT1: D1, D2, E1:; D6, E6, B5: Transgene algae transformed with the construct DGAT1 promoter:: DGAT1: E7, E9, B7, B8.

FIG. 16. is a bar graph showing fatty acids allocated to TAG in transformants derived from different constructs: nitrogen-replete (+N), or after seven days of nitrogen starvation (−N).

FIG. 17. Schematics of vector plasmids harboring short versions of promoters

FIG. 18. is a micrograph showing that EGFP is expressed and targeted to plasma membrane in P. tricornutum cells under control of shorter versions of purine permease and ammonium transporter gene promoters. LM—light microscope; EGFP—EGFP fluorescence; PAF. A-top row) p0408-1000 bp EGFP-PM; B-2^{nd} from top) p0408-515 bp EGFP-PM; C-3^{rd} from top) p8636-1000 bp EGFP-PM; D-bottom row) p8636-500 bp EGFP-PM

### DETAILED DESCRIPTION OF THE INVENTION

In one aspect, the present invention provides a novel Phaeodactylum tricornutum promoter active constitutively under nitrogen replete conditions. In another embodiment, provided herein a novel promoter active under nitrogen starvation conditions. In another embodiment, provided herein a novel promoter which is up-regulated under nitrogen starvation. In another embodiment, provided herein a novel alga promoter. In another embodiment, provided herein a novel microalga promoter. In another embodiment, provided herein a novel P. tricornutum promoter. In another embodiment, a promoter or a novel promoter of the invention may be an active fragment of a promoter as described herein. In another embodiment, an active fragment of a promoter as described herein comprises at least 500 nucleic acid residues having at least 85% identity to a promoter or a novel promoter of the invention. In another embodiment, an active fragment of a promoter as described herein comprises or consists the nucleic acid sequence of any one of SEQ ID Nos: 29-32.

In some embodiments, provided herein vectors or transformation vectors harboring algal promoters. In some embodiments, vectors of the invention are used for efficient expression and production of endogenous and foreign proteins under variable conditions and in different cellular locations, including cytoplasm (soluble), lipid droplets (hydrophobic compartment) and endomembranes (excluding chloroplast), and as secreted proteins.

In one embodiment, the promoter of the invention is DGAT1 promoter (SEQ ID NO: 1). In another embodiment, the promoter of the invention is ammonium transporter promoter (SEQ ID NO: 2). In another embodiment, the promoter of the invention is purine permease promoter (SEQ ID NO: 3).

In one embodiment, the DGAT1 promoter (SEQ ID NO: 1) is efficient in driving and/or inducing the expression of genes located 3′ to DGAT under nitrogen starvation conditions. In one embodiment, the DGAT1 promoter (SEQ ID NO: 1) is efficient in driving and/or inducing the expression of genes fused to its 3′end under nitrogen starvation conditions.

In one embodiment, the ammonium transporter promoter (SEQ ID NO: 2) is efficient in driving and/or inducing the expression of genes located 3′ to ammonium transporter promoter under nitrogen starvation conditions. In one embodiment, the ammonium transporter promoter (SEQ ID NO: 2) is efficient in driving and/or inducing the expression of genes fused to its 3′end under nitrogen starvation conditions.

In one embodiment, the purine permease promoter (SEQ ID NO: 3) is efficient in constitutively driving and/or inducing the expression of genes located 3′ to purine permease promoter. In one embodiment, the purine permease promoter (SEQ ID NO: 3) is efficient in constitutively driving and/or inducing the expression of genes fused to its 3′end.

In one embodiment, the promoter of the invention is an active fragment of the promoter of anyone of SEQ ID Nos: 1 to 3. In one embodiment, an active fragment is efficient in constitutively driving and/or inducing the expression of genes located 3′ to the active fragment. In one embodiment, an active fragment, comprises or consists the DNA of SEQ ID NO: 29-32. In one embodiment, an active fragment comprises or consists 500 consecutive nucleic acid residues derived from any of the DNA sequences recited herein (such as but not limited to SEQ ID Nos: 1-3 and/or DNA molecules derived from the primers recited herein). In one embodiment, an active fragment comprises or consists 500 nucleic acid residues having at least 80% identity to the corresponding region within a DNA sequence recited herein (such as but not limited to SEQ ID Nos: 1-3 and/or DNA molecules derived from the primers recited herein). In one embodiment, an active fragment comprises or consists 500 nucleic acid residues having at least 85% identity to the corresponding region within a DNA sequence recited herein (such as but not limited to SEQ ID Nos: 1-3 and/or DNA molecules derived from the primers recited herein). In one embodiment, an active fragment comprises or consists 500 nucleic acid residues having at least 90% identity to the corresponding region within a DNA sequence recited herein (such as but not limited to SEQ ID Nos: 1-3 and/or DNA molecules derived from the primers recited herein). In one embodiment, an active fragment comprises or consists 500 nucleic acid residues having at least 95% identity to the corresponding region within a DNA sequence recited herein (such as but not limited to SEQ ID Nos: 1-3 and/or DNA molecules derived from the primers recited herein). In one embodiment, an active fragment comprises or consists 500 nucleic acid residues having at least 97% identity to the corresponding region within a DNA sequence recited herein (such as but not limited to SEQ ID Nos: 1-3 and/or DNA molecules derived from the primers recited herein).

In some embodiments, the invention provides composite DNA sequences which comprise a promoter of the invention coupled to a gene which is not endogenically coupled to a promoter of the invention. In some embodiments, “coupled|” is connected or directly connected. In some embodiments, the invention provides composite DNA sequences which comprise a promoter of the invention located 5′ to a gene which is not 3′ to the promoter of the invention. In some embodiments, a composite DNA sequence is a vector. In some embodiments, a composite DNA sequence is a plasmid. In some embodiments, a composite DNA sequence is expressed in a cell. In some embodiments, a cell does not endogenically express a gene within a vector as described herein.

In another embodiment, a gene of interest coupled to a promoter as described herein is a transgene. In another embodiment, the invention provides a DNA molecule comprising a gene of interest coupled to a promoter. In another embodiment, the promoter of the invention is a transgenic element. In another embodiment, the promoter and the gene are derived from 2 distinct species. In another embodiment, the promoter and the gene are derived from the same species. In another embodiment, the promoter is inserted into a cell of a different species. In another embodiment, the gene is inserted into a cell of a different species-transgene. In another embodiment, provided herein a recombinant DNA molecule comprising a promoter and a gene (or a transgene) as described herein.

In another embodiment, the DGAT1 promoter comprises the nucleic acid sequence:

(SEQ ID NO: 1)
ATCAGCGTCTCGCCTCCACCTCCGTTTTGTTCCCGTACGTCATTGATTAA
ATCCGTATCAACACCGAGGGAGCCATCCCCATCACGATAGAGAATATTCA
CAAACTGATAGCCAATATTTATATCCACAAAGTCGTAAATCTTTTGAGCG
TAAACATCCGACGCTGAGACGTTCGAAACACCGTTCAATGAGACTAAAAC
TTGGTTGAATTGAAGAGATTCTCGAACATCGTCACAGTTGTTGAGACGCG
ATGGCCAGGAACCACCGTGTCGACGAATCTGTCGACGCAGTCGGGGTTTC
AATAGGGGGGAATGCTCGTCAAGGACGTGGCGTGCCAACCAGACTCGCTT
AAAAAACGGGTGGTCCGAGGCTTCGATCACCATCTTGGAAAATATCTGCT
TACTAACAAGCCTTGCCGACGGGTCTTCATCCAATGCTTGCTGATCACGG
CGATGATAACGACCCAAAAAATTTCGACTGATAACAGATGTCAACAAATC
GAATCTCGTCGATTCTGCTATGCTACTTGTGTCACTCGCAGTTTCACCTG
AAATGCTTTGGGCGGCCGAATTCCGGTCATTTTCAAAAGACTCCTGTAAG
GAGGGATCGGCATCACTCGCATCGACATTGGCAACGACATTCAGGGTTGC
GTCCATGATTTCGCCACCAACTTCATTGTAGAGTCGATTGATGATTCGAA
ATTCCAACACCGGACAGGGCTCTTTCTCTTCGTCGTCCGAAGATTCGTCG
TGCCCGTGATTGGGATCAAAATCGTTGTTTCGCCGTACCCCAGGACCAAA
CCGAATAACAATCGGATCACTAAAGATCACTTGTGCGTGTGACTGAATAC
GCAATACTTTGCCAAACAAAATTGCGCCACAAAAGCCAGAGTATAACACC
CCCAACAAAGCTTCGAGCGAGCATATAAAATTGATAAAGAAACAATGGGT
TGGGCTCGAGTTTTGGTAGCTCAAAGCTGGCGCCGTCGAACCATATCCGA
CGGTCGAAAAAGTTGTCCACGACAAGGCGAAGGCATCAGCAAAGGCTGCG
TCTGTGGACGCAAAGAGCTCTCCGCCAATTCGTACACATTCCTCATCAAT
TCTACCGGCCATGGTTATGAACCCCGCAAATAGAATCACGAGAGCAAAAA
ACATTACACACATGACCACGAACAAGAATAGAAAATTGACACGGAACATC
CAATGCAAATACATCGCAAGTTTTTGGTTGGGGTTTATGCCAATGGCGAG
ACCAGTCGAACGGCGGGGTGAGCCAATTCTTCCGTTGCACGTCGTGATTT
TCCAAAACAGAGACTGTTGAGGGCTTCGTCGGGTGGCTTCTAGACGATAC
TGCTTCGTTTGTTGATGTTGGTTCGATGCTTCCACGTCCTCCGACTGTTC
TTCGCTCTTAGAAAAAACGTTTAAAAATTTGGTTTTCAGACGTTGCCCCC
ATGGCATGGCGGTGCCTTCATCCATACGCCGAAGCTACATGTCACAAAGA
AGAATCAGCAAGTAGAATGAGTAACTGAAAAAGGAACATTTCCGCGGCCG
TTCGACTTGTTCAACTTAACCTACATGAACACATTGCGCTCGAGATATTT
GGTTTATAATGGGCTGACCGCCTTTTATATTTGACGTCATAGTTCCCGGC
TCGATCTTCGGGTTTCCCACGAAGGAATCGTAGAATACAACCGCGAACGA
TGTCTATGCGGAGCAAAAGGAAGGAGTAGCCTAAGGTTGTAAAGGGGACA
GAAACAGCAAAACGTTGGTTCACAGTCAAACGTTTCATGCCGGGTCGCGG
TTCATTTTGGTGTGTGTTTTGGAATGCGCGAGGTTGCAAACTGTGTACGT
TTATAATTTAAATACCTTTTCAAATTATAGATATTTTGACATTTCCTAAC
GTTAGTAAGTATTGCTCCGGTCTGCTTCCAACTGTAAACTCACCCTGACA
GTGAGTGAGTCTCTATTTTCTTCGCATTTTGGAGAACTCAGGCCGGACAA
AATCGTTCACTTCTTGTATAAACGGGCATCTTTATCTTACCGCTGCATTT
TCCTTCGTACAAAGAGTTAAGAGTCCAATTCTAATTTTTTTCTTGCCAGA
CAGCTAGTCCCAACAAGGAGACAGGAGGGGCTACACATACCACAGACAC
G.

In another embodiment, the DGAT1 promoter of the present invention comprises a nucleic acid sequence that is at least 60%, at least 70%, at least 80%, at least 85%, at least 90%, at least 95% or at least 98% homologous to the nucleic acid sequence of SEQ ID NO: 1. Each possibility represents a separate possibility of the invention.

In another embodiment, the DGAT1 promoter of the present invention comprises a nucleic acid sequence that is at least 60%, at least 70%, at least 80%, at least 85%, at least 90%, at least 95% or at least 98% identical to the nucleic acid sequence of SEQ ID NO: 1. Each possibility represents a separate possibility of the invention.

In another embodiment, the DGAT1 promoter as described herein comprises at least a portion of the nucleic acid provided in SEQ ID NO: 1. In another embodiment, the DGAT1 promoter as described herein is a variant of SEQ ID NO: 1.

The term “variant” as used herein refers to a DNA or a nucleic acid molecule which is derived from the sequence through the insertion or deletion of one or more nucleic acid residues or the substitution of one or more nucleic acid residues. In all cases, variants must have the DGAT1 promoter activity as defined herein.

The phrase “nucleic acid molecule” refers in some embodiments to a synthetic nucleic acid molecule, a hybrid nucleic acid molecule, a vector (such as a plasmid), a chimeric nucleic acid molecule, a recombinant nucleic acid molecule or any combination thereof.

In another embodiment, the present invention provides a protein derived from a DNA sequence regulated by the DGAT1 promoter as described herein. In another embodiment, the present invention provides a DNA molecule comprising the promoter of the invention and a gene wherein the gene is not coupled and/or regulated, endogenically, by the promoter. In another embodiment, the present invention provides a DNA molecule comprising the DGAT1 promoter and a gene wherein the gene is not coupled and/or regulated, endogenically, by the DGAT1 promoter. In another embodiment, the present invention provides a DNA molecule comprising the promoter of the invention and a gene directly connected or coupled to the promoter of the invention. In another embodiment, the present invention provides a DNA molecule comprising the promoter of the invention and a gene located 3′ to the promoter of the invention. In another embodiment, the present invention provides that the DNA molecule is a synthetic DNA molecule as the combined sequence of the promoter and the gene does not exist naturally. In another embodiment, the present invention provides that the DNA molecule is a composite DNA molecule as the combined sequence of the promoter and the gene does not exist naturally.

In another embodiment, the ammonium transporter promoter comprises the nucleic acid sequence:

(SEQ ID NO: 2)
GGGACCCAACAAAACCACCATCGATCCGATTGCAATGGCGACTAGAGGAG
GGACGGCAGCCGAAGGCACAGAGTTTTTCTTGTTGGTCAGCATGAAAATA
CAAAAGACCAAAAAAGCAGTGCCAAACGCTTCCAACAAGAAGGCGTGCCA
CCCACTTGTCACATACTTCGAAAGGCTGCGGTGCGTAAAAAGGAAAAAAT
GGACGTCAGCTTGTGTTTTGAGGAGCAAGGTTTTGGTGAAAGGCGCTTGC
AGATCCGTTGAGAAGATATTACCTCCAATAGTCGCCAAAAGCAGCTGCCG
ATTGAATTGAGGCTTGTCCCCCTCGAACAATTCCGTTCTTCGCCTCATAG
CTTAAAATGGCTTCGTGATAAATGATTAGATTTGTCAAGGCGCCGAGAAA
AGCTCCTGCCAGCTGAGCAGCCCAGTAGGGAAGGACGTTAGCGTAGGGAA
AATCAGCCGGACGAACGACAGCGAACGATAAAGTTACGGCTGGATTCAAG
TGTCCACCGGACAAGGCAGCGGACGAATAAAGACCCAAGGTGGCTCCCAG
GATCCAGACAATCGCTGCCTGCCACATGCCATCCAAGACACCGAGATAGA
TTGCTAGGCAATTGGCGCCACAACCGACTTGCACTAGAATCGCCGTCCCA
ATAAGTTCCGCCGTCAACTGAGAGCGAAGCGGGGGAGGAGGAGCATCGGT
AATTTGTGGTTCTGTTTGCGGTATGGATCCTGAAAAAGAATGACGATTGA
AGCAATTTGTATATCAGCCAGGCAATACTTCTTGGTCGCTTGTAAAGCAT
TTGCTCCTCGATTCCAAAGACGGAACGCGAACTCACCGTACTCAACCATC
TTGTCTTCTTTGTTGCAAAAACAGCTTCAGAAATTTTTGGCTCTGGATAA
GTTTACATTCGGTGCGTGTACGATCGAACTTTTACAGTAAGAACGTCGTC
CATCCTGGAGAATCGAGATCGATGTTGACTGGAAAGATCCAGGAATAATT
GTCGAGATTCGCGCTGTGCGTGACACAGATGACACTTCGCAAAACATTTT
GGCAAAAGAAGTTCGTAAAAGTTGACTTCA1GTGACATAAGGGTTACTGT
GTTTAGGGTTCGTCAAATTTTGGCACCAGAGATGACGCATGGAGCTAGAG
AAAGATCGAAGCCGTCTGAAGTGAGAATGCTCTTCTCGAAGTTATGAATA
GATTAGTGATTAGCCGATATTCCGGCAATCCCTTCACTGTGAGGCATTCA
ATTTAATTTATCTCACTGTTTGGATATCCTTTGTTGTGAAGCATTCCTTT
TGTATGGCTTTAGGTGGAAGAAAATGCTCTCCTTTGATGCTGCCGCTTGT
CGATATCAGCCCTCACCCCCAAGTCGTCAAGCCAAGATGAGTAACAGTGA
GGATTTGTACTTTCCAAAGGCTTGATCACTGGCTCATCCAAAACGATTTT
TTGTAGTGGAATCCTACGTCTCGTACTGTTGATAAGAAAAGCTATTTATG
GTCTATGGTTGAACGACGTAAGAACGGTCCTGTGAGAATTCTGTAAAACG
ACGGAGACACAAATTCAAGTGGTACTCTCTAATTATGCTGGTATAGGCCT
TGAAATGACTGACGCGCCACTGTTCTCCGACGAGCCAATCAAAACCTTTC
AATCTTACCTGGCCGTTGCCTTCTCTCGAACAATTAATCTGGAGTTCTGA
ATTCGATTTAATATTTTCTGTCTTTATCGACGCAATCTTATTGGATGACT
AAGCTTCGATAGGATTGTAGTGGCTGGAAATGTCGTTTGATTGTGCAAGC
GTGTCAATTGCGATAACAGAGTTGATGATTGGCCCAGGGCAATTATATAG
GATCAACCGTCCTGTGAAGTTGGTAATGATTCTACATACCGGAAACTGCT
ACGCTGTGCTTCTACTAAACCTTCCCTCACACCTCCTTGATCAGATCGGA
ATTTCCGTTTGCACCCTGGCGGATCCGACCTCCGTAACTTTCATAAAACT
CTGT.

In another embodiment, the isolated polynucleotide comprises a DNA sequence that is at least 60%, at least 70%, at least 80%, at least 85%, at least 90%, at least 95% or at least 98% homologous to the amino acid sequence of SEQ ID NO: 2. Each possibility represents a separate possibility of the invention.

In another embodiment, the isolated polynucleotide comprises a DNA sequence that is at least 60%, at least 70%, at least 80%, at least 85%, at least 90%, at least 95% or at least 98% identical to the amino acid sequence of SEQ ID NO: 2. Each possibility represents a separate possibility of the invention.

In another embodiment, the ammonium transporter promoter as described herein comprises at least a portion of the nucleic acid provided in SEQ ID NO: 2. In another embodiment, the ammonium transporter promoter as described herein is a variant of SEQ ID NO: 2. In all cases, variants must have the ammonium transporter promoter activity as defined herein.

In another embodiment, the present invention provides a protein derived from a DNA sequence regulated by the ammonium transporter promoter as described herein. In another embodiment, the present invention provides a DNA molecule comprising the promoter of the invention and a gene wherein the gene is not coupled and/or regulated, endogenically, by the promoter. In another embodiment, the present invention provides a DNA molecule comprising the ammonium transporter promoter and a gene wherein the gene is not coupled and/or regulated, endogenically, by the ammonium transporter promoter.

In another embodiment, the putative purine permease promoter comprises the nucleic acid sequence:

(SEQ ID NO: 3)
TACTGCAGACCGTGCCGTAGCCAAATTCGTCGTTCTGGATGAAGATGGGC
GCCAAAAGTCGTTCCAGCTCTATCCCCACGTTCGTATCCGGCAAGTGCTC
GTCTCTCGGCATTTGGGTATCGTGCAACAGATTTAACAGGTCGTCGTGAT
TCCAAGTTTCCTTGGCTTCTAAACGTACTGTTTTCTCCAACAAAGATTTC
AGACGCTCTGTTTTAGGCCAGGGCGTGTCCAACAATCCATTGCAAAGCCC
GTACACCCTCCCCGGTTCTAGAACGTGCACTTGTTGCGGATCACTTTTTG
GGTGTCGGTTGGACACGTGACACAATTGATTGCCATCGTACGCGAGTAAA
TTGAAGCCGTTGTAATCCTCACCATGCGAATGTACGCTTCGACAATACTC
GGCAGCGGACAGTTCGACGTCGGTTCGGTCGCGTTGACAAAAGCCGACGA
CCAGATCGCCGCGAGAGTGCTTGTCTTCGAATTCCTTCGGACCGCTGCGG
TGATTCGTCACGACAGCAACTCGGGATGAACCAACGCGCGCTGCTAGCCA
GCTACCACAAGACTGATCGTCCAAGCCAGCGACGAGCGCTCCATCGGACC
AAACGTGCGCTGGTCTCGAGGGTCGTGCGTAGAACTCGTCACGATTCGTA
ACGAGAATCAAGGCGTACGAGGGGTGAATGTTGACACCACAAGCGACGAT
GCACATGATGGCGTGCAGAATGCGTAATGCCGCCAGTTTCAAGAAAGTTA
GGTAATACTTATCGATTGATTGATTTACTGTTGATTCGGTTTGTGAGTTT
GTGAGGTTGCTATCCATCACCGTTTGGAGTCGATGGAGCGACAGTGAAGT
CCACGGTTCGTATCCGCTCTTGTTACCCTGTACGACTTTTGATGTGTGAT
CACGTGATGATTCTCATTCTTGAAACTGATGTAACATCAATTGTATCGAC
TGACAGTCACAGGCAGTCGATCTGGATGTCGTTTGGACACAACGAAAGCA
CGTGGTATTGTCAACTCCGGGTCTGGGGCCGCTTCTCCGGTGTCTACTCG
TGCGGTGTATGCTTCGGTACAGTGCTACAACGGATGACAGACGTGCGGCA
ATAGTTCGTTGTCCAAGTCGTTTCGAGCCTTGACATACTTTATACCAGAG
TGCCAACTTACAATTCTGTGCTATGTTTCGATGAGGTTCTTCGGACAAAA
AAATCATGGTAACAGATGCTGCTTCTCTTGTCAGTATCTAACGAAATTGA
CTAACTGTGAGAATATTACGGAGATAAACGGCGTCCCCGTACGTGGCGAA
ATGGACAAATGGGAATCGGAATTGACGGTGGGTAGGCGGCTATTTATGAA
AGAAAACCGGAACGACAGATGGAGTTGGGACAAATCAAAACTAAAATAGT
ACCATCGATAGCCAATCATTTCGAGGTGTGTCGATTCCGCATTCGTAAGT
TCTGAAGTTTTTCGGGGAAGCTGAGCCAGCGGTCGATTACCGTTAAAATA
GTATATTTTGCGACAACTTCGCTTTTCTCTATTTCTGAGCTAGTTATGCT
TTACTTGATATCATTCGTTTTTCATCGTTAAGTGATGAGTTCGTGAACGC
TGCAGCGTTTTGATCAACTTCCCTTATCAGATTGGGAGGTTGTGTTCTAG
AGTGGGATTCCCAGCAGTAGAAGTGTCGTTGATTGGCTTGGTTGAAGCCC
GGTACAGCAATGATTGGGAGGTGCCAGTGTCAAAGTTTTCCGCCACTCCA
GCCCGATATCTTTCTTCTCTCCACTACGGAATGAAGAACGGATTTTCTGC
TACTTTTTCTTTTCCCATCTGCCATAAATTTTCGTTCTGCATAATTTCAT
CGCAGTGCAACGATATTTTATACACGTTACTAATTGTTTGTCTGCTTTCT
CAACGGTTGGCAATTCGTCGTGTAAAGTACTCAAGACTATTCAATCCATT
TACGTGTGCAAGAGTTGCAACAAGAATTCCGCTCTTGTGTGAAGCATC.

In another embodiment, the isolated polynucleotide comprises a DNA sequence that is at least 60%, at least 70%, at least 80%, at least 85%, at least 90%, at least 95% or at least 98% homologous to the amino acid sequence of SEQ ID NO: 3. Each possibility represents a separate possibility of the invention.

In another embodiment, the isolated polynucleotide comprises a DNA sequence that is at least 60%, at least 70%, at least 80%, at least 85%, at least 90%, at least 95% or at least 98% identical to the amino acid sequence of SEQ ID NO: 3. Each possibility represents a separate possibility of the invention.

In another embodiment, the putative purine permease promoter as described herein comprises at least a portion of the nucleic acid provided in SEQ ID NO: 3. In another embodiment, the putative purine permease promoter as described herein is a variant of SEQ ID NO: 3. In all cases, variants must have the putative purine permease promoter activity as defined herein.

In another embodiment, the present invention provides a protein derived from a DNA sequence regulated by the putative purine permease promoter as described herein. In another embodiment, the present invention provides a DNA molecule comprising the putative purine permease promoter of the invention and a gene wherein the gene is not coupled and/or regulated, endogenically, by the promoter. In another embodiment, the present invention provides a DNA molecule comprising the putative purine permease promoter and a gene wherein the gene is not coupled and/or regulated, endogenically, by the putative purine permease promoter.

In another embodiment, the present invention provides a method for producing a protein encoded by a gene of interest. In another embodiment, the present invention provides a method for producing a plant or an algal protein encoded by a gene of interest. In another embodiment, the present invention provides a method for producing a plant or an algal protein encoded by a gene of interest, wherein the quantity of the protein is rate limiting in the biosynthesis of PUFA and/or ARA.

In another embodiment, the present invention provides a method for producing a protein of interest, comprising ligating a gene of interest to a promoter of the invention, wherein the promoter is not the endogenous promoter of the gene of interest, further comprising the step of transforming a cell with the ligated polynucleotide. In another embodiment, a polynucleotide or a DNA molecule as described herein comprises a promoter as described herein from one source/species and a gene from another source/species. In another embodiment, the invention provides a vector comprising polynucleotide or a DNA molecule as described herein. In another embodiment, the invention provides a plasmid comprising a polynucleotide or a DNA molecule as described herein. In another embodiment, the invention provides an agrobacterium comprising polynucleotide or a DNA molecule as described herein.

In another aspect, the present invention provides a protein resulting from a polynucleotide or a DNA molecule as described herein. In another aspect, the present invention provides that the gene encodes a polypeptide. In another aspect, the present invention provides that the gene encodes an enzyme.

In another embodiment, the method of the present invention further comprises the step of transforming a cell with at least one polynucleotide comprising a promoter as described herein and a gene encoding an enzyme involved in the biosynthesis of ω-3 LC-PUFA. In another embodiment, the method of the present invention further comprises the step of transforming a cell with a promoter as described herein and a gene encoding: ω-3 desaturase, C20 PUFA elongase, Δ4 desaturase or combinations thereof.

In another embodiment, the expression and/or transcription of the gene encoding the enzyme involved in the biosynthesis of ω-3 LC-PUFA as described herein is up-regulated by the promoter of the invention during nitrogen starvation. In another embodiment, the expression and/or transcription of ω-3 desaturase, C20 PUFA elongase and Δ4 desaturase as described herein, is constitutively up-regulated by the promoter of the invention.

In another embodiment, a long-chain polyunsaturated fatty acid produced by a protein or a combination of proteins encoded by genes coupled to promoters as described herein in a cell or alga cell produced by methods described herein, is utilized in an infant formula. In another embodiment, a long-chain polyunsaturated fatty acid produced by a protein or a combination of proteins in a cell or alga cell produced by methods described herein, is administered to a subject having a deficiency in very long-chain polyunsaturated fatty acid.

In another embodiment, a protein as described herein is an enzyme which participates in the biosynthesis of a fatty acid. In another embodiment, a protein as described herein is an enzyme which participates in the biosynthesis of long-chain polyunsaturated fatty acid (LC-PUFA). In another embodiment, a protein as described herein is an enzyme which participates in the biosynthesis of arachidonic acid (ARA). In another embodiment, a protein as described herein is an enzyme which participates in the biosynthesis of triacylglycerols (TAG). In another embodiment, a protein as described herein is an enzyme which participates in the biosynthesis of ω-3 fatty acid. In another embodiment, a protein as described herein is an enzyme which participates in the biosynthesis of ARA. In another embodiment, a protein as described herein is an enzyme which participates in the biosynthesis of eicosapentaenoic acid. In another embodiment, a protein as described herein is an enzyme which participates in the biosynthesis of docosahexaenoic acid.

In another embodiment, a protein as described herein is a desaturase. In another embodiment, a protein as described herein is a ω-3 desaturase. In another embodiment, a protein as described herein is an elongase. In another embodiment, a protein as described herein is C20 PUFA elongase. In another embodiment, a protein as described herein is Δ4 desaturase

In another embodiment, a cell is a eukaryotic cell. In another embodiment, a cell is a prokaryotic cell. In another embodiment, a cell is a plant cell. In another embodiment, a cell is an algal cell. In another embodiment, a cell is a microalga cell. In another embodiment, a cell is a Parietochloris incisa cell. In another embodiment, a cell is a transfected cell. In another embodiment, a cell is transiently transfected with a polynucleotide or a combination of polynucleotides as described herein. In another embodiment, a cell is stably transfected cell with a polynucleotide or a combination of polynucleotides as described herein.

In another embodiment, the present invention provides a composition comprising a vector comprising a polynucleotide as described herein. In another embodiment, the present invention provides composition comprising a combination of vectors which comprise polynucleotides as described herein. In another embodiment, a composition such as described herein, comprises an excipient. In another embodiment, a composition such as described herein, comprises a carrier. In another embodiment, a carrier stabilizes a protein or a nucleic acid molecule of the invention. In another embodiment, one of skill in the art will readily identify a known suitable carrier to be used with the composition as described herein.

In another embodiment, algae as described herein are eukaryotic organisms. In another embodiment, algae as described herein are photoautotrophic. In another embodiment, algae as described herein are mixotrophic. In another embodiment, algae as described herein are unicellular. In another embodiment, algae as described herein are multicellular. In another embodiment, algae as described herein are Excavata algae. In another embodiment, algae as described herein are Rhizaria algae. In another embodiment, algae as described herein are Chromista algae. In another embodiment, algae as described herein are Alveolata algae.

Polypeptides and Polynucleotides

In some embodiments, the terms “protein” or “polypeptide” are used herein interchangeably and encompasses native polypeptides (either degradation products, synthetically synthesized polypeptides or recombinant polypeptides) and peptidomimetics (typically, synthetically synthesized polypeptides), as well as peptoids and semipeptoids which are polypeptide analogs, which have, in some embodiments, modifications rendering the polypeptides/proteins even more stable while in a body or more capable of penetrating into cells.

In some embodiments, modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein.

In some embodiments, polypeptide bonds (—CO—NH—) within the polypeptide are substituted. In some embodiments, the polypeptide bonds are substituted by N-methylated bonds (—N(CH3)-CO—), by ester bonds (—C(R)H—C—O—O—C(R)—N—), by ketomethylene bonds (—CO-CH2-) or α-aza bonds (—NH—N(R)—CO—), wherein R is any alkyl, e.g., methyl, carbo bonds (—CH2-NH—). Each possibility represents a separate embodiment of the invention. In some embodiments, the polypeptide bonds are substituted by hydroxyethylene bonds (—CH(OH)—CH2-), by thioamide bonds (—CS—NH—), by olefinic double bonds (—CH═CH—) or by retro amide bonds (—NH—CO—), by polypeptide derivatives (—N(R)—CH2-CO—), wherein R is the “normal” side chain, naturally presented on the carbon atom. In some embodiments, these modifications occur at any of the bonds along the polypeptide chain and even at several (2-3 bonds) at the same time.

In some embodiments, natural aromatic amino acids of the polypeptide such as Trp, Tyr and Phe, be substituted for synthetic non-natural acid such as Phenylglycine, TIC, naphthylelanine (Nol), ring-methylated derivatives of Phe, halogenated derivatives of Phe or o-methyl-Tyr. In some embodiments, the polypeptides of the present invention include one or more modified amino acid or one or more non-amino acid monomers (e.g., fatty acid, complex carbohydrates, etc.).

In one embodiment, “amino acid” or “amino acids” is understood to include the 20 naturally occurring amino acid; those amino acid often modified post-translationally in vivo, including, for example, hydroxyproline, phosphoserine and phosphothreonine; and other unusual amino acid including, but not limited to, 2-aminoadipic acid, hydroxylysine, isodesmosine, nor-valine, nor-leucine and ornithine. In one embodiment, “amino acid” includes both D- and L-amino acid.

In some embodiments, the polypeptides or proteins of the present invention are utilized in a soluble form. In some embodiments, the polypeptides or proteins of the present invention include one or more non-natural or natural polar amino acid, including but not limited to serine and threonine which are capable of increasing polypeptide or protein solubility due to their hydroxyl-containing side chain.

In some embodiments, the polypeptides or proteins of the present invention are utilized in a linear form, although it will be appreciated by one skilled in the art that in cases where cyclization does not severely interfere with polypeptide characteristics, cyclic forms of the polypeptide can also be utilized.

In some embodiments, recombinant protein techniques utilizing the promoters of the invention are used to generate the polypeptides of the present invention. In some embodiments, recombinant protein techniques utilizing the promoters of the invention are used for generation of relatively long polypeptides (e.g., longer than 18-25 amino acid). In some embodiments, recombinant protein techniques utilizing the promoters of the invention are used for the generation of large amounts of the polypeptide of the present invention. In some embodiments, recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al, (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

In one embodiment, a polypeptide or protein of the present invention is synthesized using a polynucleotide encoding a polypeptide or protein of the present invention coupled to the promoter of the invention. In another embodiment, coupled is operably coupled. In another embodiment, coupled is operably linked. In another embodiment, coupled is contiguously coupled. In another embodiment, the promoter is located 5′ to the gene to which it is coupled to or ligated to. In some embodiments, the promoter of the invention is a cis-regulatory sequence. In some embodiments, the promoter (such as SEQ ID NO: 3) is suitable for directing constitutive expression of the gene of interest.

In one embodiment, the phrase “a polynucleotide” refers to a single or double stranded nucleic acid sequence which may be isolated and provided in the form of an RNA sequence, a complementary polynucleotide sequence (cDNA), a genomic polynucleotide sequence and/or a composite polynucleotide sequences (e.g., a combination of the above). Each possibility represents a separate embodiment of the invention. In one embodiment, “genomic polynucleotide sequence” refers to a sequence derived (isolated) from a chromosome and thus it represents a contiguous portion of a chromosome.

In one embodiment, “composite polynucleotide sequence” refers to a DNA molecule as described herein comprising a gene of interest such as a gene encoding a protein involved in fatty acid production coupled or ligated to a promoter as described herein. In one embodiment, a composite sequence can include some exonal sequences required to encode the polypeptide of the present invention, as well as some intronic sequences interposing there between. In one embodiment, the intronic sequences can be of any source, including of other genes, and typically will include conserved splicing signal sequences. In one embodiment, intronic sequences include cis acting expression regulatory elements.

In one embodiment, the polynucleotides of the present invention further comprise a signal sequence encoding a signal peptide for the secretion of the polypeptides of the present invention. In one embodiment, following expression, the signal peptides are cleaved from the precursor proteins resulting in the mature proteins.

In some embodiments, polynucleotides of the present invention are prepared using PCR techniques or any other method or procedure known to one skilled in the art. In some embodiments, the procedure involves the legation of two different DNA sequences (See, for example, “Current Protocols in Molecular Biology”, eds. Ausubel et al., John Wiley & Sons, 1992).

In one embodiment, polypeptides or proteins of the present invention are purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In one embodiment, the polypeptide or protein of the present invention is retrieved in “substantially pure” form. In one embodiment, the phrase “substantially pure” refers to a purity that allows for the effective use of the protein in the applications described herein.

In one embodiment, the polypeptide or protein of the present invention can also be synthesized using in vitro expression systems. In one embodiment, in vitro synthesis methods are well known in the art and the components of the system are commercially available.

Expression and Transformation Systems

In another embodiment, the terms “transformation”, “transduction”, “transfection” and “conjugation” are used herein interchangeably and refer to the insertion of new genetic material into nonbacterial cells including animal and plant cells, applied to eukaryotic and prokaryotic cells.

In another embodiment, the present invention provides an engineered organism such as a transgenic plant, a transgenic seed, a transgenic alga and a transgenic animal. Each possibility represents a separate embodiment of the invention. In another embodiment, an engineered organism is engineered to express a protein, a polypeptide, a combination of polypeptides, a polynucleotide and a combination of polynucleotides as described herein. Each possibility represents a separate embodiment of the invention.

In one embodiment, polynucleotides of the present invention are inserted into expression vectors (i.e., a nucleic acid construct) to enable expression of the recombinant polypeptide. In one embodiment, the expression vector of the present invention includes additional sequences which render this vector suitable for replication and integration in prokaryotes. In one embodiment, the expression vector of the present invention includes additional sequences which render this vector suitable for replication and integration in eukaryotes. In one embodiment, the expression vector of the present invention includes a shuttle vector which renders this vector suitable for replication and integration in both prokaryotes and eukaryotes. In some embodiments, cloning vectors comprise transcription and translation initiation sequences (e.g., promoters, enhancers) and transcription and translation terminators (e.g., polyadenylation signals).

In one embodiment, a variety of prokaryotic or eukaryotic cells can be used as host-expression systems to express the polypeptides of the present invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In some embodiments, non-bacterial expression systems are used (e.g., plant expression systems) to express the polypeptide of the present invention. In one embodiment, yeast expression systems are used. In one embodiment, algae expression systems are used. In one embodiment, plant expression systems are used. In one embodiment, a number of vectors containing constitutive or inducible promoters can be used in yeast as disclosed in U.S. Pat. No. 5,932,447 which is hereby incorporated in its entirety by reference. In another embodiment, vectors which promote integration of foreign DNA sequences into the yeast chromosome are used.

In another embodiment, expression in a host cell can be accomplished in a transient or a stable fashion. In another embodiment, a host cell is a cell as described herein. In another embodiment, transient expression is from introduced constructs which contain expression signals functional in the host cell, but which constructs do not replicate and rarely integrate in the host cell, or where the host cell is not proliferating. In another embodiment, transient expression also can be accomplished by inducing the activity of a regulatable promoter operably linked to the gene of interest.

In another embodiment, stable expression is achieved by introduction of a construct of a polynucleotide of the invention that integrates into the host genome. In another embodiment, stable expression additionally comprises autonomously replication within the host cell. In another embodiment, stable expression of the polynucleotide of the invention is selected for the use of a selectable marker located on or transfected with the expression construct, followed by selection for cells expressing the marker. In another embodiment, the site of the construct's integration can occur randomly within the host genome or can be targeted through constructs containing regions of homology with the host genome sufficient to target recombination with the host locus. In another embodiment, constructs are targeted to an endogenous locus, all or some of the transcriptional and translational regulatory regions can be provided by the endogenous locus.

In another embodiment, the regeneration, development, and cultivation of plants from single plant protoplast transformants or from various transformed explants is well known in the art (for example: Weissbach and Weissbach, In: Methods for Plant Molecular Biology, (Eds.), Academic Press, Inc. San Diego, Calif., (1988)). In another embodiment, regeneration and growth process comprises the steps of selection of transformed cells, culturing those individualized cells through the usual stages of embryonic development through the rooted plantlet stage. In another embodiment, transgenic embryos and seeds are similarly regenerated. In another embodiment, resulting transgenic rooted shoots are thereafter planted in an appropriate plant growth medium such as soil.

In another embodiment, regeneration and growth process of algae are known to one of skill in the art. In another embodiment, identification, selection, of transgenic algae are known to one of skill in the art.

In another embodiment, development or regeneration of plants containing an exogenous polynucleotide as described herein encodes a protein as described herein and is well known in the art. In another embodiment, development or regeneration of algae containing an exogenous polynucleotide as described herein encodes a protein as described herein and is well known in the art. In another embodiment, the regenerated plants are self-pollinated to provide homozygous transgenic plants. In another embodiment, pollen obtained from the regenerated plants is crossed to seed-grown plants of agronomically important lines. In another embodiment, pollen from plants of these important lines is used to pollinate regenerated plants. In another embodiment, a transgenic plant of the present invention containing a desired polypeptide is cultivated using methods well known to one skilled in the art.

In another embodiment, a variety of methods can be utilized for the regeneration of plants from plant tissue. In another embodiment, the method of regeneration will depend on the starting plant tissue and the particular plant species to be regenerated. In another embodiment, methods for transforming dicots, primarily by use of Agrobacterium tumefaciens, and obtaining transgenic plants are known in the art McCabe et al., Biol. Technology 6:923 (1988), Christou et al., Plant Physiol. 87:671-674 (1988)); Cheng et al., Plant Cell Rep. 15:653657 (1996), McKently et al., Plant Cell Rep. 14:699-703 (1995)); Grant et al., Plant Cell Rep. 15:254-258, (1995).

In another embodiment, transformation of monocotyledons using electroporation, particle bombardment, and Agrobacterium are known in the art. In another embodiment, transformation and plant regeneration are well established in the art. In another embodiment, assays for gene expression based on the transient expression of cloned nucleic acid constructs have been developed by introducing the nucleic acid molecules into plant cells by polyethylene glycol treatment, electroporation, or particle bombardment (Marcotte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goff et al., EMBO J. 9:2517-2522 (1990)).

In another embodiment, transient expression systems are used to functionally dissect the oligonucleotides constructs. In another embodiment, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see for example, Sambrook et al., Molecular Cloning: A Laboratory Manual, Cold Spring Harbor Press (1989); Maliga et al., Methods in Plant Molecular Biology, Cold Spring Harbor Press (1995); Birren et al., Genome Analysis:

Detecting Genes, 1, Cold Spring Harbor, N.Y. (1998); Birren et al., Genome Analysis: Analyzing DNA, 2, Cold Spring Harbor, N.Y. (1998); Plant Molecular Biology: A Laboratory Manual, eds. Clark, Springer, N.Y. (1997)).

In one embodiment, the expression vector of the present invention can further include additional polynucleotide sequences that allow, for example, the translation of several proteins from a single mRNA such as an internal ribosome entry site (IRES) and sequences for genomic integration of the promoter-chimeric polypeptide.

In some embodiments, expression vectors containing regulatory elements as described herein and possibly from eukaryotic viruses such as retroviruses are used by the present invention.

In one embodiment, plant expression vectors are used. In one embodiment, the expression of a polypeptide coding sequence is driven by a number of promoters including at least one promoter as described herein. Other expression systems such as insects and mammalian host cell systems, which are well known in the art, can also be used by the present invention.

It will be appreciated that other than containing the necessary elements for the transcription and translation of the inserted coding sequence (encoding the polypeptide or protein), the expression construct of the present invention can also include sequences engineered to optimize stability, production, purification, yield or activity of the expressed polypeptide or protein.

In some embodiments, transformed cells are cultured under effective conditions, which allow for the expression of high amounts of recombinant polypeptide or protein (such as nitrogen starvation (SEQ IS NOs: 1 and 2)). In some embodiments, effective culture conditions include, but are not limited to, effective media, bioreactor, temperature, pH and oxygen conditions that permit protein production. In one embodiment, an effective medium refers to any medium in which a cell is cultured to produce the recombinant polypeptide or protein of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, depending on the vector and host system used for production, resultant polypeptides or proteins of the present invention either remain within the recombinant cell, secreted into the fermentation medium, secreted into a space between two cellular membranes, or retained on the outer surface of a cell or viral membrane.

Some examples of substances which can serve as carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the compound is basically determined by the way the compound is to be administered. If the subject compound is to be injected, in one embodiment, the pharmaceutically-acceptable carrier is sterile, physiological saline, with a blood-compatible suspending agent, the pH of which has been adjusted to about 7.4.

In addition, the compositions further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., aspartame, citric acid), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

The compositions also include incorporation of the proteins or oligonucleotides of the invention into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In some embodiments, the proteins or oligonucleotides of the invention modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. In another embodiment, the modified proteins or oligonucleotides of the invention exhibit substantially longer half-lives in blood following intravenous injection than do the corresponding unmodified compounds. In one embodiment, modifications also increase the proteins or oligonucleotides solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Materials and Methods for Example 1

Culture Conditions

Axenic cultures of P. tricornutum, strain 646, were obtained from the Culture Collection of Algae and Protozoa (CCAP) at the Scottish Marine Institute (SAMS Research Services Ltd, Oban, UK). P. tricornutum cultures were cultivated on RSE medium in an incubator shaker under enrichment of 200 cc·min⁻¹ of CO₂ and controlled temperature (18° C.) and illumination (50 μmol photons m⁻² s⁻¹) at a speed of 120 rpm. RSE medium was composed of 34 g L⁻¹ Reef Salt (Seachem, Madison, Ga., USA) with nutrient supplementation as described by Guihéneuf et al. (2011). To induce nitrogen starvation, daily-diluted cultures were centrifuged (1200 g for 5 min), washed twice, and resuspended in nitrogen free RSE medium. The nitrogen-free medium was prepared by omitting KNO₃ from the RSE medium. Cultures were further maintained under the same conditions, except the illumination (150 μmol photons m⁻² s⁻¹). Samples for cell counting, dry weight determination, TAG and fatty acid (FA) analysis, RNA extraction for quantitative real-time PCR (qRT-PCR) analysis and protein extraction for Western analysis were removed periodically.

Plasmids Construction
PCR amplifications was carried out with PhusionHot Start II DNA polymerase (Finnzymes, Finland)
Primers were designed with the Primer Design tool for In-Fusion HD Cloning Kit.
Ligations were performed using the In-Fusion HD Cloning Kit (Clontech, Calif.).
The plasmid pPHA-T1::2 (Zaslayskaia et al. 2000), was used as the backbone for all constructed vectors. pPHA-T1 refers to the original plasmid (Zaslayskaia et al. 2000), including the fcp-A promoter region.
A full list of all oligonucleotide primers used in this work can be found in Table 1.

Promoters Used in Vector Constructs for Transformation of P. tricornutum

The following genes ammonium transporter (GI:219120408), putative purine permease (GI:219128636), DGAT1 (GI:325073417), actin/actin like protein ACT2 (GI: 219123832) were selected for their promoters.

pPHA-T1::2 was digested with StuI and XbaI in order to remove 2. The sequence for the signal peptide, targeted to plasma membrane (PM), was employed for the plasma membrane marking by using 25 amino acids adopted from the farnesylation signal from c-Ha-Ras (Aronheim et al. 1994) and a stop codon was added (Primer 1 containing a StuI restriction site (underlined) and 2). The farnesylation signal peptide (SGLRSKLNPPDESGPGCMSCKCVLS (SEQ ID NO: 4)) was not amplified, instead oligo annealing was used in order to get double-stranded DNA. The signal peptide was cloned right after the PCR product of 2 (2 without stop codon) (Primers 3 and 4 containing a XbaI restriction site (underlined)), and named pPHA-T1::GFP-memb-sp.

The plasmids: pPHA-T4::p8636-GFP-Memb-sp, pPHA-T4-p0408-GFP-Memb-sp, pPHA-T4-P.t Act2-GFP-Memb-sp and pPHA-T4-P.t DGAT1-GFP-Memb-sp were constructed in several stages. The first step, in order to insert all promoters, pPHA-T1: 2-Memb-sp, was digested with NdeI and StuI in order to remove the fcp-A promoter. The promoters sequence (2000 bp upstream to ATG of all genes) obtained from JGI (http://genome.jgi-psf.org/Phatr2/Phatr2.home.html), was amplified from P. tricornutum DNA and ligated into final plasmids. Finally, before transformations all plasmids were sent for sequencing at the DNA Microarrays and Sequencing Laboratory, BGU

Microparticle Bombardment

Transformation of P. tricornutum was carried out as described by Zaslayskaia et al. (2000), with minor modification. P. tricornutum cells grown in 5% CO₂ under continuous illumination were harvested at the mid-logarithmic phase. Approximately 2.5-5×10⁸ cells/mL were plated onto RSE agar plate (1.5%). 1 mg of gold microcarriers (0.6 μm diameter particle size) was coated with 2 μg of plasmid DNA in the presence of 2.5M CaCl₂) and 0.1M spermidine. The PDS-1000/He biolistic particle delivery system (Bio-Rad, Hercules, Calif.) was used for microprojectile bombardments. The bombardment was performed at 1350 psi under a negative pressure of 27 inches mercury with a target distance of 6 cm. Bombarded cells were illuminated for 24 hours. ˜1×10⁷ cells each were then were plated onto solid medium containing 100 μg·mL⁻¹ Zeocin. The plates were placed under constant illumination (75 μmol photons·m⁻² s⁻¹) for 4-6 weeks.

Colonies Screening

After 4-6 weeks when colonies have appear on the selection plates, screening for positive colonies was done in a 96 wells (Edge BioSystems, USA). To each well a volume of 200 μl RSE (with nitrogen) was added. Each colony from the Zeocin plates was replaced into each well. After 2 weeks in an incubator shaker under enrichment of 200 cc/min of CO2 and controlled temperature (18° C.) and illumination (50 μmol photons m⁻² s⁻¹) at a speed of 120 rpm, to induce nitrogen starvation, cultures were centrifuged (1200 g for 5 min), and resuspended in nitrogen free RSE medium, then the cells were incubated under these conditions for 14 days, and then observed under fluorescence microscopy every day.

Fluorescence Microscopy

For basic screening and microscopic observations were done under bright and fluorescence light with AxioScope with HBO50, Carl Zeiss MicroImaging Inc. and documented with an Olympus DP70 camera, and with a Zeiss Axio Imager A2 fluorescence microscope coupled with an AxioCam. Images were acquired with ZEN imaging software, with Zeiss GFP set 38HE and Filter set 16 shift free (F) filters.

Zeiss LSM 510 Meta was used to capture in vivo localizations of fluorescent proteins. For 2, A 488-nm laser was used for the excitation of both GFP and chlorophyll, and the received fluorescent light was split using a NFT565 beam splitter, and was detected simultaneously in two channels with BP500-550, and LP650 filters, respectively.

RNA Isolation and Synthesis of cDNA

Isolation of mRNA was performed using the ZR Plant RNA MiniPrep kit (Zymo Research, USA) following manufacturer's instructions. Total RNA concentration was measured by a NanoDrop 1000 Spectrophotometer (Thermo Fisher Scientific, USA) and calculated using the NanoDrop software, 3.0.0 (Coleman Technologies Inc., USA). The RNA integrity was estimated by formaldehyde gel electrophoresis.

Template cDNA for qRT-PCR was isolated from 10 mL of culture. The cells were harvested by centrifugation at 1000 g for 5 min, washed twice with 20 mM HEPES and 150 mM NaCl (pH 7) and flash-frozen in liquid nitrogen, and stored at −80 C° until further use. Total RNA samples were treated with RapidOut DNA Removal Kit (Thermo Scientific, Lithuania) according to manufacturer's protocol, before being used for cDNA synthesis. cDNA was prepared from 500 ng of total RNA-template in total volume of 10 μl with blend of random hexamers and anchored oligo-dT 3:1 (v/v) by the Verso cDNA kit, (Thermo scientific, Lithuania).

DNA Isolation

DNA isolation from P. tricornutum was done as described by Doyle (Doyle 1987)

qRT-PCR Primer Design and Validation:

qRT-PCR primer pairs were designed for 2 (Zaslayskaia et al. 2000) and the housekeeping (HKGs) gene GI-219124034 (predicted protein, Clathrin), qRT-PCR primer pairs were designed for all relevant genes with PrimerQuest (http://eu.idtdna.com/PrimerQuest/Home/Index). The putative HKGs analyzed in this work was chosen based on it constant expression under nitrogen starvation conditions based on the expressed sequence tag (EST) dataset (Maheswari et al. 2010). This included predicted melting temperatures (Tm) of 60±2° C., primer lengths of 20-24 nucleotides, and PCR amplicon lengths of 140-180 bp. Primers were designed according to these criteria using the Netprimer program (http://www.premierbiosoft.com/netprimer/). Amplification of single products of the expected sizes was verified on 2% (w/v) agarose gels. The nucleotide sequences and characteristics of primers used for qRT-PCR analysis are presented in

qRT-PCR Conditions and Statistical Analysis

qRT-PCR conditions and statistical analysis was done as described by Siaut et al (2007) with minor modification; Primer pairs were validated with five serial beginning with 5-fold dilutions of cDNA samples and primers. Standard curves were plotted to test for linearity of the response. The primer pairs and primer concentrations with reaction efficiencies of 100%±10% were chosen for qRT-PCR analysis of relative gene expression. Gene expression profiling was performed by qRT-PCR, using triplicate reactions for each sample of two independent RNA isolations with a gene-specific primer pair and SsoFast EvaGreen Supermix (Bio-Rad, Hercules, Calif., USA), in a CFX96 Real-Time System (Bio-Rad). The amplification procedure was 95° C. for 30 s, 40 cycles of 95° C. for 5 s, and 55° C. for 5 s. A melting curve was obtained for each pair of primers to confirm that a single, specific product was produced in each reaction.

Real-Time qPCR Data Visualization

The results are presented as the relative expression normalized to the expression of endogenous genes that were stable under the experimental conditions and used as house-keeping gene predicted protein, Clathrin (GI:219124034). All quantifications were performed by the CFX 42 Manager™ Software (Bio-Rad, USA). Real-time qPCR procedures and analyses were performed in accordance with the MIQE guidelines (Bustin et al. 2009).

Protein Electrophoresis

For Western blot analysis, total proteins were extracted from 20 ml of cultured cells, harvested by centrifugation at 3000 g for 5 min and re-suspended in 250 μl of in 20 mM HEPES (pH 7.5) containing 150 mM NaCl. 2.5 mm glass beads, were added and the cells, supplemented with 1 μl protease inhibitor cocktail (Sigma-Aldrich, USA), were broken in the bead beater three times sequentially for one minute each and cooled on ice for one minute between repetitions. The supernatant was then transferred to a new 2 ml tube. Protein concentration was determined by the Bradford reagent (BioRad, USA). Proteins were extracted by adding sample buffer (125 mM Tris-HCl 4% SDS, 20% glycerol, 10% mercaptoethanol, 0.004 bromophenol blue, pH 6.8) for a final concentration of 1 μg/μl of protein and incubated for one hour at room temperature. Twenty micrograms of total proteins were resolved by SDS/PAGE blotted onto nitrocellulose membrane 2 μm (BioRad, USA). For visualization of proteins on the SDS gels, a GelCode Blue Stain Reagent (Thermo scientific, Lithuania) was used. For visualization of proteins on the nitrocellulose membrane Ponceau S (Sigma-Aldrich, USA) was used. 2 was detected by incubation with Anti-Tag(CGY)FP antibody (Evrogen, Russia) at a dilution of 1:5,000, and with goat anti-rabbit alkaline phosphatase conjugate with HRP (BioRad, USA) at a dilution of 1:3,000. The membrane was developed with an ECL chemiluminescence, EZ-ECL Kit (Biological Industries, Israel) detection system according to company instructions and documented with a MicroChemi imager (DNR Bio-Imaging 16 Systems Ltd.).

### Materials and Methods for Example 2

Strain and Culture Conditions

Axenic cultures of P. tricornutum, strain 646, were obtained from the Culture Collection of Algae and Protozoa (CCAP) at the Scottish Marine Institute (SAMS Research Services Ltd, Oban, UK). P. tricornutum was cultivated on RSE medium in an incubator shaker under enrichment of 200 cc min⁻¹ of CO₂ and controlled temperature (18° C.) and illumination (50 μmol photons m⁻² s⁻¹) at a speed of 120 rpm. RSE medium was composed of 34 g L⁻¹ Reef Salt (Seachem, Madison, Ga., USA) with modification as described by Guihéneuf et al. (2011). To induce nitrogen starvation, daily-diluted cultures were centrifuged (1200 g for 5 min), washed twice, and resuspended in nitrogen-free RSE medium. The nitrogen-free medium was prepared by omitting KNO₃ from the RSE medium. Cultures were further maintained under the same conditions. Samples for cell counting, RNA extraction for qRT-PCR analysis and protein extraction for Western Blot analysis were withdrawn periodically. The growth parameters (cell number, chlorophyll content and dry weight) were determined essentially as described in Pal et al., 2013; for the dry weigh determination, cells were washed with five volumes of ammonium formate (3%, w/v).

Plasmid Design and Construction

PCR amplifications have been carried out with PhusionHot Start II DNA polymerase (Finnzymes, Finland). Primers were designed with the Primer Design tool for In-Fusion HD Cloning Kit. The ligation was made by In-Fusion HD Cloning Kit (Clontech, Calif.). The plasmid pPHA-T1::EGFP (Zaslayskaia et al., 2000), was used as the backbone for all constructed vectors (kindly provided by Prof. P. Kroth), carrying the fcpA promoter region for expression of recombinant genes. A full list of oligonucleotide primers used in this work can be found in Table 1.

The plasmids pPHA-T1::eGFP-HOGP and pPHA-DGAT1::eGFP-HOGP were constructed in several stages. The first step involved in creating pPHA-DGAT1::2. In order to create pPHA-DGAT1:: eGFP, pPHA-T1::eGFP was digested with NdeI and EcoRI in order to remove the fcp-A promoter. The DGAT1 promoter sequence, (GI:325073417)-2000 bp upstream to ATG—was obtained from JGI (http://genome.jgi-psf.org/Phatr2/Phatr2.home.html), amplified from P. tricornutum DNA and ligated into final plasmids. PCR amplification was carried out with the forward primer (1) containing a NdeI restriction site (underlined), and the reverse primer (2) an EcoRI restriction site (underlined). The second step involved cutting pPHA-DGAT1::eGFP with StuI and SphI, in order to remove eGFP, followed by insertion of the eGFP (EGFP without stop codon) and HOGP genes simultaneously. Those were produced as follows: 2 was amplified with the forward primer (3) containing a StuI restriction site (underlined), and the reverse primer (4), HOGP was amplified using the forward primer (5) and the reverse primer (6), containing a SphI restriction site (underlined). To build pPHA-T1::eGFP-HOGP, pPHA-T1::eGFP was digested with StuI+XbaI, in order to remove eGFP, followed by re-ligation of eGFP fused to HOGP, that were amplified by using pPHA-DGAT1::eGFP-HOGP as template using the forward primer (7) containing a StuI restriction site (underlined), and the reverse primer (8), containing an XbaI restriction site (underlined).

Diatom Transformation

Transformation of P. tricornutum was carried out as described by Zaslayskaia et al. (2000), with minor modification. P. tricornutum cells grown in the incubator shaker in 2% CO₂-enriched atmosphere under continuous illumination as described above were harvested at the mid-logarithmic phase. Approximately 2.5-5×10⁸ cells/ml were plated on the surface of the RSE agar plate (1.5%). One mg of gold microcarriers (6 μm diameter particle size, Bio-Rad, USA) was coated with 5 μg of plasmid DNA in the presence of 2.5M CaCl₂) and 0.1M spermidine. Vectors were introduced into P. tricornutum by microparticle bombardment using a Biolistic PDS-1000/He Particle Delivery System (Bio-Rad, USA). The bombardment was performed at 1350 psi under a negative pressure of 27 inches mercury with a target distance of 6 cm. Bombarded cells on the agar plates were illuminated with white light (75 μmol m⁻² s⁻¹) at 18° C. for 24 hours; cells were recovered by resuspension in liquid RSE and re-plated onto solid medium containing 100 μg·mL⁻¹ zeocin (˜1×10⁷ cells per plate). The plates were placed under constant illumination (75 μmol photons/m²·s⁻¹) at 18° C. for 4-6 weeks to allow transgenic colonies to appear.

Colonies Screening

After 4-6 weeks when colonies appeared on the selection plates, screening for positive colonies was done in 96 well plates (50063, Edge BioSystems, USA). To each well a volume of 200 μl RSE (with nitrogen source) was added. After 2 weeks in an incubator shaker under enrichment of 200 cc/min of CO₂ and controlled temperature (18° C.) and illumination (50 μmol photons/m² s) at a speed of 120 rpm, each well was supplemented with palmitoleic acid (C16:1, Nu-Check Prep), as free fatty acid (FFA), to a final concentration of 100 μg/ml (400 μM) (from stock of 100 mg/ml in DMSO). Cell cultures were incubated under these conditions for at least 16 hours, and then were observed by fluorescence microscopy.

DNA, RNA Isolation and Synthesis of cDNA

DNA isolation from P. tricornutum was done as described by Doyle (Doyle, 1987). For total RNA isolation, the cells were harvested by centrifugation at 1000 g for 5 min, washed twice with 20 mM HEPES and 150 mM NaCl (pH 7) and flash-frozen in liquid nitrogen, and stored at −80 C° until further use. Total RNA was isolated using the ZR Plant RNA MiniPrep kit (Zymo Research, USA) following manufacturer's instructions. Total RNA samples were treated with RapidOut DNA Removal Kit (Thermo Scientific, Lithuania) according to manufacturer's protocol, before being used for cDNA synthesis. Total RNA concentration was measured by a NanoDrop 1000 Spectrophotometer (Thermo Fisher Scientific, USA) and calculated using the NanoDrop software, 3.0.0 (Coleman Technologies Inc., USA). The RNA integrity was estimated by formaldehyde gel electrophoresis. cDNA was prepared from 500 ng of total RNA-template in total volume of 10 μl with blend of random hexamers and anchored oligo-dT 3:1 (v/v) by the Verso cDNA kit (Thermo scientific, Lithuania).

Gene Expression Analysis

Gene expression analysis was performed by qRT-PCR, using triplicate reactions for each sample of two independent RNA isolations with a gene-specific primer pair and SsoFast EvaGreen Supermix (Bio-Rad, Hercules, Calif., USA), in a CFX96 Real-Time System (Bio-Rad). The amplification procedure was 95° C. for 30 s, 40 cycles of 95° C. for 5 s, and 55° C. for 5 s. A melting curve was obtained for each pair of primers to confirm that a single, specific product was produced in each reaction.

qRT-PCR primer pairs were designed for all relevant genes (Table 1) with PrimerQuest (http://eu.idtdna.com/PrimerQuest/Home/Index). Primer pairs were validated with five serial 5-fold dilutions of cDNA samples and primers. Standard curves were plotted to test for linearity of the response. The primer pairs and primer concentrations with reaction efficiencies of 100%±10% were chosen for qRT-PCR analysis of relative gene expression. The nucleotide sequences and characteristics of primers used for qRT-PCR analysis are presented in Table 2.

Western Blot Analysis of Transgene Protein Expression

For Western blot analysis, total proteins were extracted from 20 ml of cultured cells harvested by centrifugation at 3000 g for 5 min and re-suspended in 20 mM HEPES containing 150 mM NaCl. Glass beads (2.5 mm in diameter) were added and the cells, supplemented with protease inhibitor cocktail (Sigma-Aldrich, USA) and 10 mM Tris/Cl (pH 7.5) were broken by bead beating three times sequentially for one minute at a time and cooled on ice for one min between repetitions. Proteins were extracted by adding lysis buffer (195 mM Tris-HCl 15% SDS, 45% glycerol, 6% mercaptoethanol, pH 6.8) to cell pellets, for a final concentration of 20 μg/μl, and cell lysates were incubated for one hour at room temperature. Protein concentration was determined by the Bradford reagent (Bio-Rad, USA). Twenty micrograms of total proteins were resolved by SDS/PAGE blotted onto 2 μm nitrocellulose membrane (162-0112, BioRad, USA). For visualization of proteins on the SDS gels, a GelCode Blue Stain Reagent (Thermo scientific, Lithuania) was used. For visualization of proteins on the nitrocellulose membrane Ponceau S (Sigma Aldrich, USA) was used. EGFP was detected by incubation with Anti-Tag(CGY)FP antibody (Evrogen, Russia) at a dilution of 1:5,000, and with goat anti-rabbit alkaline phosphatase conjugate with HRP (BioRad, USA) at a dilution of 1:3,000. The membrane was developed with an ECL chemiluminescence, EZ-ECL Kit (Biological Industries, Israel) detection system according to manufacturer's instructions and documented with a MicroChemi imager (DNR Bio-Imaging 16 Systems Ltd.).

Fluorescence Microscopy

Basic screening and microscopic observations were done by bright-field and fluorescence microscopy with a Zeiss Axio Imager A2 fluorescence microscope (Carl Zeiss MicroImaging Inc.) equipped with an AxioCam. Images were acquired with ZEN imaging software, with filter sets 38 HE and 16 used for visualization and documentation of eGFP and chlorophyll autofluorescence, respectively.

A confocal microscope Zeiss LSM 510 Meta was used to capture in vivo localizations of fluorescent proteins. For eGFP, a 488-nm laser was used for the excitation of both GFP and chlorophyll, and the received fluorescent light was split using a NFT565 beam splitter, and was detected simultaneously in two channels with BP500-550, and LP650 filters, respectively.

Cultivation of P. tricornutum Transformant Lines for Induction of TAG Production

Lipid and fatty acid analyses were performed in the nitrogen-depleted cultures cultivated in 100 ml Erlenmeyer flasks as described above. The pre-cultures were grown in complete RSE medium under light intensity of 100 μmole photons/m² s at 25° C. with periodical dilution to chlorophyll concentration of 10 mg L⁻¹. To induce nitrogen starvation, cells were pelleted by centrifugation, washed three times in nitrogen-free RSE and reconstituted in nitrogen-free medium to the initial Chl content of 15 mg L⁻¹ (˜1×10⁷ cell/ml).

Lipid Extraction and Fatty Acid Analysis

Prior to lipid extraction, P. tricornutum biomass samples, freeze-dried and kept at −20° C., were treated for 10 min with hot isopropanol at 80° C. to prevent lipase activity. Following centrifugation, isopropanol was collected, and lipids were extracted from the remaining cell pellets according to the method of Bligh and Dyer (Bligh and Dyer, 1959) and combined with isopropanol. TAG was isolated from total lipid extract by TLC on Silica Gel 60 plates (Merck, USA) using a solvent system of petroleum ether: diethyl ether: acetic acid (70:30:1, v/v/v).

A fatty acid analysis by GC was performed on dried biomass samples, and TAG recovered from TLC by extraction with chloroform: methanol (1:1, v/v) mixtures. Prior to the direct transmethylation of dried biomass, 5 ml of cultures were pelleted by centrifugation, and the cell pellets were dried for 45 min at room temperature in a SpeedVac concentrator (Thermo Fisher Scientific, USA) or overnight in a vacuum freeze dryer. Dried samples were incubated for transmethylation in dry methanol containing 2% (v/v) H₂SO₄ at 80° C. for 1.5 h under an argon atmosphere and continuous stirring. Pentaenoic (C15:0; Sigma-Aldrich) was added as an internal standard. Fatty acid methyl esters (FAMEs), formed as a result of the transmethylation procedure, were analyzed on a Trace GC Ultra (Thermo, Italy) equipped with a flame ionization detector (FID) and a programmed temperature vaporizing (PTV) injector (Pal et al., 2013).

Statistical Analyses

Statistical analyses were conducted using IMP version 10 (SAS Institute, USA). Analysis of variance (ANOVA) was used to identify overall significant differences between treatments. When significant differences were found, mean separations were calculated using a Tukey post hoc test. The significance level was P≤0.05.

### Example 1—Characterization of P. tricornutum Promoters Under Different Conditions

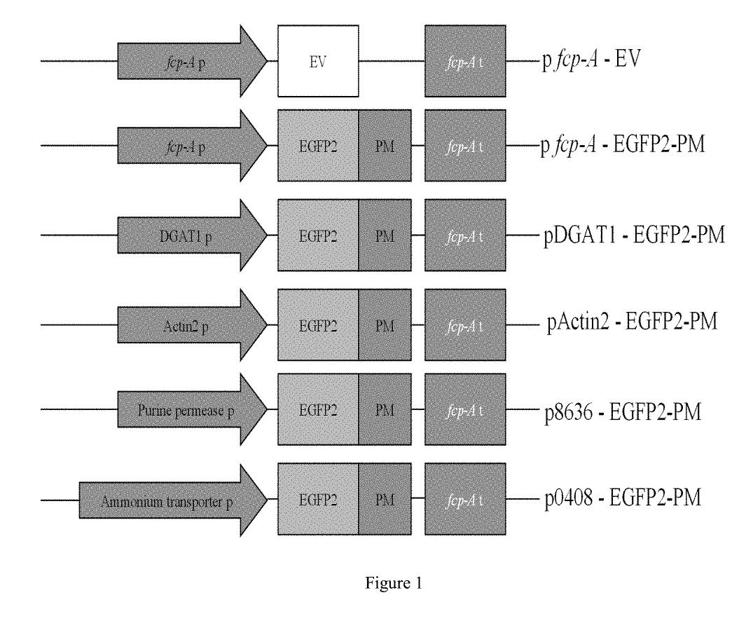

Constructs containing a EGFP-plasma-membrane recognition signal fusion protein, under control of five different promoter sequences, but terminated by the same 3′ fcp-A UTR, were designed, whereby the novel promoters were selected either based on own expression analysis (Guihéneuf et al. 2011) or based on published EST and transcriptomics expression data (Maheswari et al. 2005). Promoters showing especially high expression under nitrogen starvation conditions were selected, and 2000 bp each were cloned replacing the original fcp-A promoter of the construct. The architecture of those constructs is shown in FIG. 1.

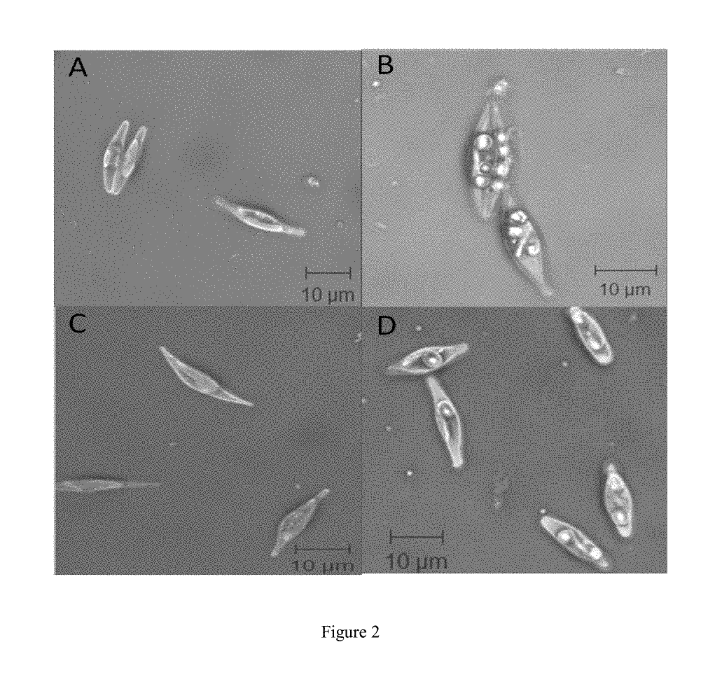

Those constructs were transformed into P. tricornutum (strain 646), and Zeocin resistant colonies were picked and cultivated. Presence of the transgenes was confirmed by PCR amplification, using PCR-primers (list) specific for gene junctions not found in wild type DNA (result not shown). Subsequently several lines of each strain were analysed by fluorescence microscopy, and strains showing representative GFP fluorescence signals were cultivated for further analysis of the GFP expression patterns, by transferring cells to nitrogen starvation (HOW?) and observation during seven days of starvation. Significant differences in GFP expression were immediately dependent on different promoters and length of starvation (FIG. 2): fcp-A promoter showed very high GFP expression at time zero, that rapidly decreased and became barely visible after one day of starvation, and completely disappeared after three and seven days of starvation. The Actin2 promoter revealed relatively weak GFP intensities except for day seven of starvation, where no signal was detected; The DGAT 1 promoter revealed weak GFP fluorescence after 3 and 7 days of starvation, and none before; Purine permease promoter revealed significant GFP fluorescence at time zero, that strengthened significantly after 3 and seven days of starvation; Ammonium transporter promoter produced barely detectable fluorescence at time zero, strengthened to maximal fluorescence after 3 days of starvation, and then seemed to decrease at day seven (see FIG. 2).

GFP-PM Expression Analysis by Western Blot

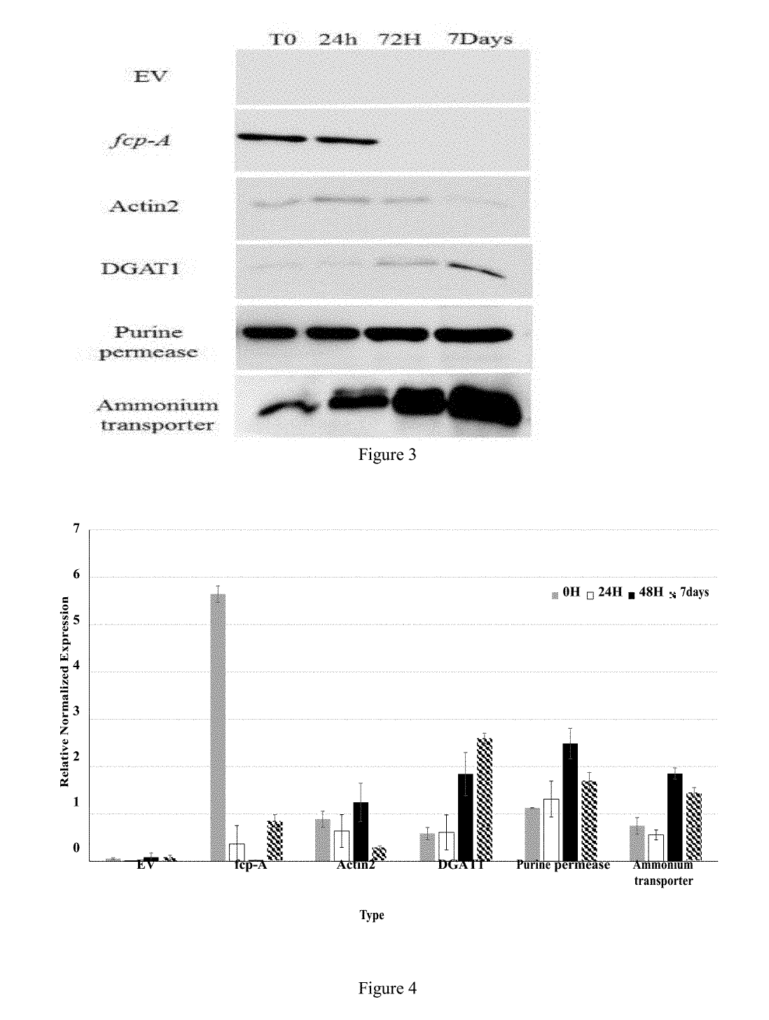

Those results were confirmed by visualizing the amount of GFP protein in the cells by Western Blot analysis using anti-GFP antibodies. The results observed by fluorescence microscopy were essentially confirmed by the Western blot analysis. However, the far more quantitative evaluation by this method indicates that the fcp-A promoter induced GFP accumulation only at days 0-1 of starvation. The two newly isolated promoters, purine permease and ammonium transporter promoters, produced an estimated order of magnitude more protein, than the actin or DGAT1 promoters. Purine permease revealed a very constitutive expression pattern, while ammonium transporter promoter was clearly and strongly induced by nitrogen starvation conditions (FIG. 3). Similarly, the actin promoter revealed rather constitutive, but weak GFP expression, while the DGAT1 promoter induced protein expression best late in starvation.

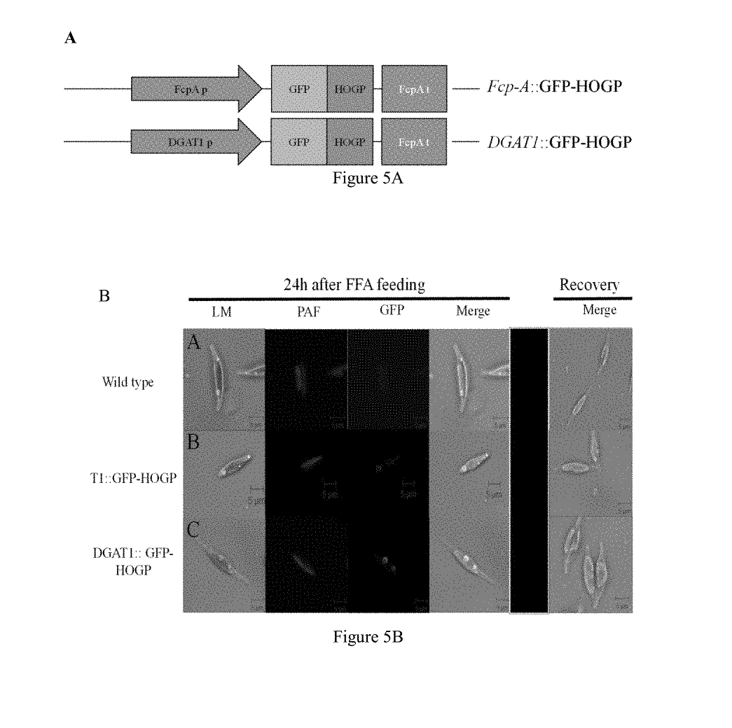

An interesting result was obtained when comparing mRNA levels and protein levels for the GFP-PM fusion protein under the different conditions with different promoters (FIG. 4). Under control of the fcp-A promoter the transgene mRNA was strongly expressed at time zero, mRNA level rapidly decreased already at day one of starvation and then completely disappeared, though after seven days a small signal reappeared. Under control of the actin2 promoter low mRNA levels were observed until day 3, which strongly decreased at day 7. Under control of the DGAT1 promoter very low mRNA levels were observed at days 0 and 1 that strongly increased at days 3 and seven, indicating that this is a late starvation induced promoter, as confirmed before (Guihéneuf et al. 2011). Under control of purine permease promoter significant transcript levels, but lower than those observed with the fcp-A promoter, were observed at all times. However, mRNA levels were never higher than those achieved with the DGAT1 promoter, though the Western Blot signal indicative for protein amount was dramatically stronger. This indicates that the promoter sequence used efficiently stimulates post transcriptional mechanisms enhancing translation and protein accumulation, apparently by signals located in the 5′ UTR of the cloned promoter sequence. The same seems true for ammonium transporter promoter which revealed strong inducability of protein production with nitrogen starvation. mRNA levels remained relatively low increasing only 2-3 fold between days zero and seven, though the amount of protein accumulated increased by orders of magnitude. Post-transcriptional mechanisms must be postulated, including stimulation of translation under nitrogen starvation, as well as inhibition of translation under nitrogen replete conditions.

Fatty acid and lipid biosynthesis in microalgae are key for enhancing productivities of many economically important products. Many microalgae produce significant amounts of oil (triacylglycerides, TAG) under stress conditions (Hu et al. 2008; Rodolfi et al. 2009). Production of TAG is widely investigated in the framework of developing technologies for the production of algal biofuels, specifically biodiesel. The model algae P. tricornutum is a species of interest for the production of biofuels and high value products. Although the full sequence of the genome of P. tricornutum has been available since 2008 (Bowler et al. 2008), and a number of effective tools are available for its genetic engineering, promoters effectively expressing proteins under nitrogen starvation conditions have not been described so far. Both the fcp-A (Apt et al. 1994) and the nitrate reductase promoter isolated from C. fuciformis (Poulsen & Kroger 2005) which are the most widely used promoters in P. tricornutum, are rarely reported to be used under nitrogen starvation. Rosenwasser et al. (2014) reported the use of the histone 4 (H4) promoter under nitrogen starvation conditions within 72 hours of starvation, but not at longer cultivation times. Sakaue et al. (2008) described the use of various viral promoters with variable success, while another report described iron-responsive promoters (Yoshinaga et al. 2014), but there were no indications that those promoters are able to drive extrinsic genes under nitrogen starvation conditions. Two of the promoters described by Sakaue et al. (2008) were tested, but did not achieve visible expression of GFP. In order to maximize TAG productivity, a stringent nitrogen starvation protocol leading to immediate rearrangement of cellular metabolism, was developed. In order to express transgenes under those conditions, genes expressing under complete nitrogen depletion were used.

The present invention provides isolation and characterization of several endogenous P. tricornutum promoters selected based on their high expression specifically under nitrogen starvation. These results clearly represent the difference between promoters able to increase their expression under nitrogen starvation conditions (ammonium transporter and DGAT1), or to maintain a constant high level of expression under nitrogen starvation conditions (purine permease). The DGAT1 promoter showed good inducibility and high GFP expression after prolonged nitrogen starvation (in accordance with expression analysis of DGAT1 mRNA, Guiheneuf et al. (2011), while the Actin2 promoter showed rather weak protein expression under starvation. In contrast, the fcp-A promoter which is strongest under nitrogen replete conditions, is dramatically down regulated already after one day, and completely silenced after 3 days of nitrogen starvation.

Moreover, very strong and clear GFP expression was detected, on the plasma membrane, even after more than 30 days under nitrogen starvation combined with high light conditions in both ammonium transporter and purine permease promoters (Data no shown). Purine permease was characterized as a constitutive promoter, while ammonium transporter promoters was found to be induced mostly under nitrogen starvation.

All the HKGs that were tested, under nitrogen replete conditions, such as that suggested by Siaut et al. (2007), were down regulated drastically under these conditions, and could not be used as reliable HKGs. The same phenomenon was observed when actin and tubulin were used as the HKG. Clathrin (GI-219124034) was chosen as the HKG, based on its relatively stable expression Maheswari et al. (2010) and proved to be the most suitable. It should be noted that the qPCR result shown in FIG. 4 is affected by this issue. A surprising result was obtained when comparing the mRNA levels for the EGFP-PM transcription under nitrogen starvation conditions (FIG. 4) with the apparent amounts of protein accumulated as determined by Western blots. Although ammonium transporter and purine permease promoters, were both upregulated under nitrogen starvation conditions, mRNA level remained relatively low and little changed comparable to that obtained using the DGAT1 promoter, and significantly below the levels observed using the fcp-A promoter under nitrogen replete conditions. This phenomenon is best seen for ammonium transporter promoter: mRNA levels increased by only 2-3 fold between day zero and seven, though the amount of protein accumulated increased by orders of magnitude. This indicates that this promoter or 5′ UTR sequence promotes highly efficient post transcriptional stimulation of protein accumulation, apparently by signals having a strong stimulatory effect on translational activity possibly regulated by stress related signals. Also protein accumulation driven by the purine permease promoter was far higher than that achieved by the DGAT 1 promoter for example, though mRNA levels were similar. The fact that post-transcriptional signals can strongly regulate protein accumulation has been well characterized in C. reinhardtii under light-dark conditions and other organisms and is apparently mediated by proteins binding to the 5′ UTR of the mRNA (Danon & Mayfield 1994). Thus, this mechanism might be regulated by nitrogen starvation induced factors is however new and of great interest for studying stress related control of algal metabolism.

TABLE 1

| | | | Promoter | |
|---|---|---|---|---|
| Name | | | Amplicon | |
| of | Primer | | osize | Protein |
| plasmid | name | Sequence (5′ -to 3′) | (bp) | (Promoter) I |
| p | | AATTCGATCGTCAGGCCTATGGTG | | |
| PHA-T1:: | | AGCAAGGGCGAGGAGCTGT (SEQ ID NO: 5) | | |
| GF | | AGATCGAGTCCGGACTTGTACAG | | |
| P-MEMB- | | CTCGTCCATGCCGA (SEQ ID NO: 6) | | |
| SP | | | | |
| p | | TCCGGACTCAGATCTAAGCTGAAC | | |
| PHA-T1:: | | CCTCCTGATGAGAGTGGCCCCGGCTGCA | | |
| GF | | TGAGCTGCAAGTGTGTGCTCTCCTGATCT | | |
| | | AGAGTCGACCTGC (SEQ ID NO: 7) | | |
| P-MEMB- | | GCAGGTCGACTCTAGATCAGGAG | | |
| SP | | AGCACACACTTGCAGCTCATGCAGCCGG | 435 | 95362 |
| | | GGCCACTCTCATCAGGAGGGTTCAGCTT | | |
| | | AGATCTGAGTCCGGA (SEQ ID NO: 8) | | |
| p | | TGAGAGTGCACCATATGTACTGCA | | |
| PHA- | | GACCGTGCCGTAGCC (SEQ ID NO: 9) | | |
| T4- | | CTTGCTCACCATAGGCCTGATGCT | 2000 | 19128636 |
| P8636- | | TCACACAAGAGCGGAA (SEQ ID NO: 10) | | |
| GFP- | | | | |
| MEMB- | | | | |
| SP | | | | |
| p | | TGAGAGTGCACCATATGGGGACC | | |
| PHA- | | CAACAAAACCAC (SEQ ID NO: 11) | | |
| T4- | | CTTGCTCACCATAGGCCTACAGAG | 2000 | 19120408 |
| P0408- | | TTTTATGAAAGTTACG (SEQ ID NO: 12) | | |
| GFP- | | | | |
| MEMB- | | | | |
| SP | | | | |
| p | | TGAGAGTGCACCATATGTGACAG | | |
| PHA- | | ATAGCTTTCAAAAG (SEQ ID NO: 13) | | |
| T4-P.T | | CTTGCTCACCATAGGCCTGATGGG | 2000 | 19123832 |
| ACT2- | 0 | ATAGAGAGGTGTT (SEQ ID NO: 14) | | |
| GFP- | | | | |
| MEMB- | | | | |
| SP | | | | |
| p | 1 | TGAGAGTGCACCATATGATCAGCG | | |
| PHA- | | TCTCGCCTCCACCTCCGT (SEQ ID NO: 15) | | |
| T4-P.T | 2 | CTTGCTCACCATAGGCCTCGTGTC | 2000 | 25073417 |
| DGAT1- | | TGTGGTATGTGTA (SEQ ID NO: 16) | | |
| GFP- | | | | |
| MEMB- | | | | |
| SP | | | | |

| Name | Primer | Sequence | Amplicon | Protein |
|---|---|---|---|---|
| of genes | name | (5′ -to 3′) | size (bp) | GI |
| predicted | q-LD1 | CGTG | 151 | 219124034 |
| protein, | fw | GATGATGA | | |
| Clathrin | | GGACTACG | | |
| | | AATC (SEQ | | |
| | | ID NO: 17) | | |
| | q-LD1 | CCAC | | |
| | rv | TTCTTATTG | | |
| | | CGACGGTA | | |
| | | AAC (SEQ | | |
| | | ID NO: 18) | | |
| 2 | q-2 fw | AAGG | 172 | 807408573 |
| | | ACGACGGC | | |
| | | AACTACAA | | |
| | | (SEQ ID NO: | | |
| | | 19) | | |
| | | TCTG | | |
| | | CTTGTCGGC | | |
| | | CATGATAT | | |
| | | AG (SEQ ID | | |
| | | NO: 20) | | |

Shorter Versions of Purine Permease and Ammonium Transporter Gene Promoters

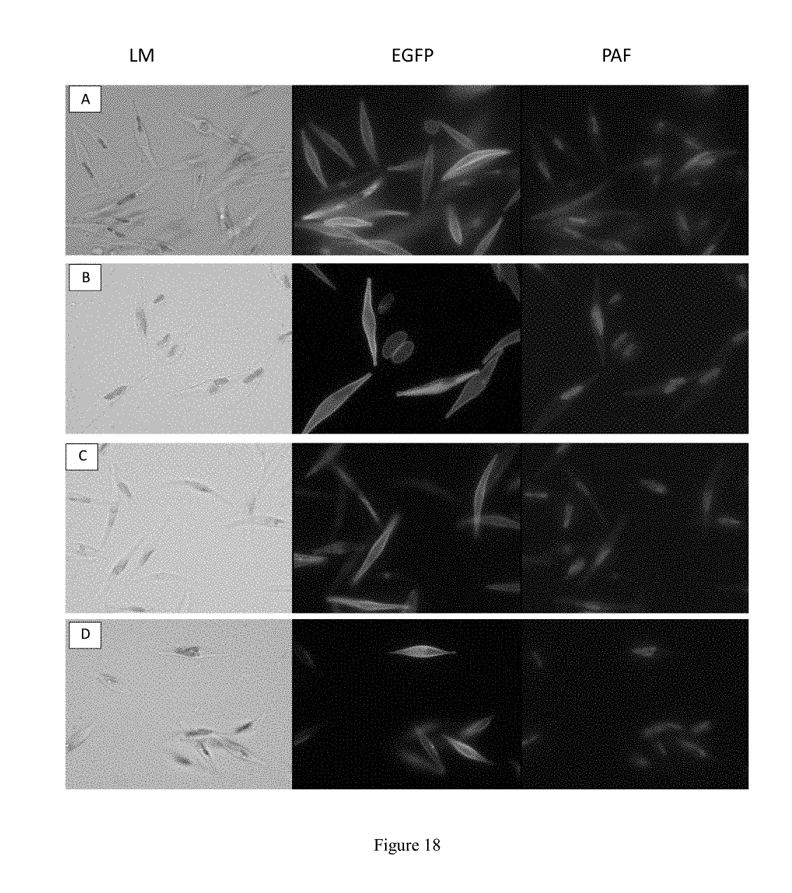

In order to construct vectors, harboring the shorter versions of purine permease ammonium transporter gene promoters, the plasmid PHA-T1:: EGFP-PM (pfcp-A::EGFP-Memb-sp), was digested with NdeI and StuI in order to remove the fcp-A promoter. The respective shorter promoter sequences (1000 bp and 500 bp upstream of putative purine permease gene and 1000 bp and 515 bp of Ammonium transporter gene) were amplified from P. tricornutum DNA with primers specified in Table 4 and ligated into final plasmids (FIG. 17). EGFP-PM signal was successfully expressed in P. tricornutum cells (FIG. 18).

TABLE 4
Oligonucleotide primers used to clone shorter versions of promoters

| | | | Promoter |
|---|---|---|---|
| Plasmid | Primer | Sequence (5′ -to 3′) | size (bp) |
| T5-p8636- | F | TGAGAGTGCACCATATGCACGTGGTATT | 1000 |
| 1000 bp | | GTCAACTC (SEQ ID NO: 21) | |
| EGFP-PM | R | CTTGCTCACCATAGGCCTGATGCTTCACA | |
| | | CAAGAGCG (SEQ ID NO: 22) | |
| T5-p0408- | F | TGAGAGTGCACCATATGGAGATTCGCGC | 1000 |
| 1000 bp | | TGTGCGT (SEQ ID NO: 23) | |
| EGFP-PM | R | CTTGCTCACCATAGGCCTACAGAGTTTTA | |
| | | TGAAAGTTACG (SEQ ID NO: 24) | |
| T5-p8636- | F | TGAGAGTGCACCATATGTAGTATATTTTG | 500 |
| 500 bp | | CGACAACTTC (SEQ ID NO: 25) | |
| EGFP-PM | R | CTTGCTCACCATAGGCCTGATGCTTCACA | |
| | | CAAGAGCG (SEQ ID NO: 26) | |
| T5-p0408- | F | TGAGAGTGCACCATATGAGCTATTTATGG | 515 |
| 515 bp | | TCTATGGTT (SEQ ID NO: 27) | |
| EGFP-PM | R | CTTGCTCACCATAGGCCTACAGAGTTTTA | |
| | | TGAAAGTTACG (SEQ ID NO: 28) | |

The DNA Sequences of the successful shorter purine permease (SEQ ID Nos: 29 and 31) and ammonium transporter gene promoters SEQ ID Nos: 30 and 32):

T5-p8636-1000bp-EGFP-PM (short)
(SEQ ID NO: 29)
CACGTGGTATTGTCAACTCCGGGTCTGGGGCCGCTTCTCCGGTGTCTACT
CGTGCGGTGTATGCTTCGGTACAGTGCTACAACGGATGACAGACGTGCGG
CAATAGTTCGTTGTCCAAGTCGTTTCGAGCCTTGACATACTTTATACCAG
AGTGCCAACTTACAATTCTGTGCTATGTTTCGATGAGGTTCTTCGGACAA
AAAAATCATGGTAACAGATGCTGCTTCTCTTGTCAGTATCTAACGAAATT
GACTAACTGTGAGAATATTACGGAGATAAACGGCGTCCCCGTACGTGGCG
AAATGGACAAATGGGAATCGGAATTGACGGTGGGTAGGCGGCTATTTATG
AAAGAAAACCGGAACGACAGATGGAGTTGGGACAAATCAAAACTAAAATA
GTACCATCGATAGCCAATCATTTCGAGGTGTGTCGATTCCGCATTCGTAA
GTTCTGAAGTTTTTCGGGGAAGCTGAGCCAGCGGTCGATTACCGTTAAAA
TAGTATATTTTGCGACAACTTCGCTTTTCTCTATTTCTGAGCTAGTTATG
CTTTACTTGATATCATTCGTTTTTCATCGTTAAGTGATGAGTTCGTGAAC
GCTGCAGCGTTTTGATCAACTTCCCTTATCAGATTGGGAGGTTGTGTTCT
AGAGTGGGATTCCCAGCAGTAGAAGTGTCGTTGATTGGCTTGGTTGAAGC
CCGGTACAGCAATGATTGGGAGGTGCCAGTGTCAAAGTTTTCCGCCACTC
CAGCCCGATATCTTTCTTCTCTCCACTACGGAATGAAGAACGGATTTTCT
GCTACTTTTTCTTTTCCCATCTGCCATAAATTTTCGTTCTGCATAATTTC
ATCGCAGTGCAACGATATTTTATACACGTTACTAATTGTTTGTCTGCTTT
CTCAACGGTTGGCAATTCGTCGTGTAAAGTACTCAAGACTATTCAATCCA
TTTACGTGTGCAAGAGTTGCAACAAGAATTCCGCTCTTGTGTGAAGCATC
T5-p0408-1000bp-EGFP-PM(short)
(SEQ ID NO: 30)
GAGATTCGCGCTGTGCGTGACACAGATGACACTTCGCAAAACATTTTGGC
AAAAGAAGTTCGTAAAAGTTGACTTCAGTGACATAAGGGTTACTGTGTTT
AGGGTTCGTCAAATTTTGGCACCAGAGATGACGCATGGAGCTAGAGAAAG
ATCGAAGCCGTCTGAAGTGAGAATGCTCTTCTCGAAGTTATGAATAGATT
AGTGATTAGCCGATATTCCGGCAATCCCTTCACTGTGAGGCATTCAATTT
AATTTATCTCACTGTTTGGATATCCTTTGTTGTGAAGCATTCCTTTTGTA
TGGCTTTAGGTGGAAGAAAATGCTCTCCTTTGATGCTGCCGCTTGTCGAT
ATCAGCCCTCACCCCCAAGTCGTCAAGCCAAGATGAGTAACAGTGAGGAT
TTGTACTTTCCAAAGGCTTGATCACTGGCTCATCCAAAACGATTTTTTGT
AGTGGAATCCTACGTCTCGTACTGTTGATAAGAAAAGCTATTTATGGTCT
ATGGTTGAACGACGTAAGAACGGTCCTGTGAGAATTCTGTAAAACGACGG
AGACACAAATTCAAGTGGTACTCTCTAATTATGCTGGTATAGGCCTTGAA
ATGACTGACGCGCCACTGTTCTCCGACGAGCCAATCAAAACCTTTCAATC
TTACCTGGCCGTTGCCTTCTCTCGAACAATTAATCTGGAGTTCTGAATTC
GATTTAATATTTTCTGTCTTTATCGACGCAATCTTATTGGATGACTAAGC
TTCGATAGGATTGTAGTGGCTGGAAATGTCGTTTGATTGTGCAAGCGTGT
CAATTGCGATAACAGAGTTGATGATTGGCCCAGGGCAATTATATAGGATC
AACCGTCCTGTGAAGTTGGTAATGATTCTACATACCGGAAACTGCTACGC
TGTGCTTCTACTAAACCTTCCCTCACACCTCCTTGATCAGATCGGAATTT
CCGTTTGCACCCTGGCGGATCCGACCTCCGTAACTTTCATAAAACTCTGT
T5-p8636-500bp-EGFP-PM (short)
(SEQ ID NO: 31)
TAGTATATTTTGCGACAACTTCGCTTTTCTCTATTTCTGAGCTAGTTATG
CTTTACTTGATATCATTCGTTTTTCATCGTTAAGTGATGAGTTCGTGAAC
GCTGCAGCGTTTTGATCAACTTCCCTTATCAGATTGGGAGGTTGTGTTCT
AGAGTGGGATTCCCAGCAGTAGAAGTGTCGTTGATTGGCTTGGTTGAAGC
CCGGTACAGCAATGATTGGGAGGTGCCAGTGTCAAAGTTTTCCGCCACTC
CAGCCCGATATCTTTCTTCTCTCCACTACGGAATGAAGAACGGATTTTCT
GCTACTTTTTCTTTTCCCATCTGCCATAAATTTTCGTTCTGCATAATTTC
ATCGCAGTGCAACGATATTTTATACACGTTACTAATTGTTTGTCTGCTTT
CTCAACGGTTGGCAATTCGTCGTGTAAAGTACTCAAGACTATTCAATCCA
TTTACGTGTGCAAGAGTTGCAACAAGAATTCCGCTCTTGTGTGAAGCATC
T5-p0408-515bp EGFP-PM (short)
(SEQ ID NO: 32)
AGCTATTTATGGTCTATGGTTGAACGACGTAAGAACGGTCCTGTGAGAAT
TCTGTAAAACGACGGAGACACAAATTCAAGTGGTACTCTCTAATTATGCT
GGTATAGGCCTTGAAATGACTGACGCGCCACTGTTCTCCGACGAGCCAAT
CAAAACCTTTCAATCTTACCTGGCCGTTGCCTTCTCTCGAACAATTAATC
TGGAGTTCTGAATTCGATTTAATATTTTCTGTCTTTATCGACGCAATCTT
ATTGGATGACTAAGCTTCGATAGGATTGTAGTGGCTGGAAATGTCGTTTG
ATTGTGCAAGCGTGTCAATTGCGATAACAGAGTTGATGATTGGCCCAGGG
CAATTATATAGGATCAACCGTCCTGTGAAGTTGGTAATGATTCTACATAC
CGGAAACTGCTACGCTGTGCTTCTACTAAACCTTCCCTCACACCTCCTTG
ATCAGATCGGAATTTCCGTTTGCACCCTGGCGGATCCGACCTCCGTAACT
TTCATAAAACTCTGT

### Example 2—DGAT1 Promoter is Effective in Enhancing Production of a Protein of Interest

A foreign green algal major LD protein, HOGP, fused to were expressed in P. tricornutum, using two different, endogenous promoters. An important objective of this work was the expression of HOGP under conditions triggering LDs formation in order to examine its localization to LDs and resulting impact on TAG accumulation in a microalgal model organism of So far, proteomics data are not available for the LDs' proteome of P. tricornutum. Adequate tools for the recombinant expression of LDs proteins specifically under nutrient stress were also missing. Furthermore, the present protein similarity searches in the P. tricornutum genome did not identify any significant similarity to the previously identified or annotated MLDPs of green algae or eustigmatophytes.

EGFP-HOGP Localizes to Lipid Droplets in the Diatom P. tricornutum

In order to overexpress the GFP-HOGP fusion protein in P. tricornutum, a comparative analysis of two gene promoters (FIG. 5A) under two distinctive assay conditions, was performed. To drive expression of HOGP the promoter region of the P. tricornutum DGAT1 gene containing 2000 bp upstream of the ATG triplet was used; this gene promoter is strongly inducible in vivo upon transfer to medium lacking nitrogen that induces intensive LDs formation and TAG accumulation (Guihéneuf et al., 2011). Another construct bearing the fcpA (fucoxanhin chlorophyll binding protein A) promoter was used for comparison (FIG. 5A); this strong promoter for a photosynthesis-related gene is widely explored for expression studies in Phaeodactylum under nutrient replete conditions.

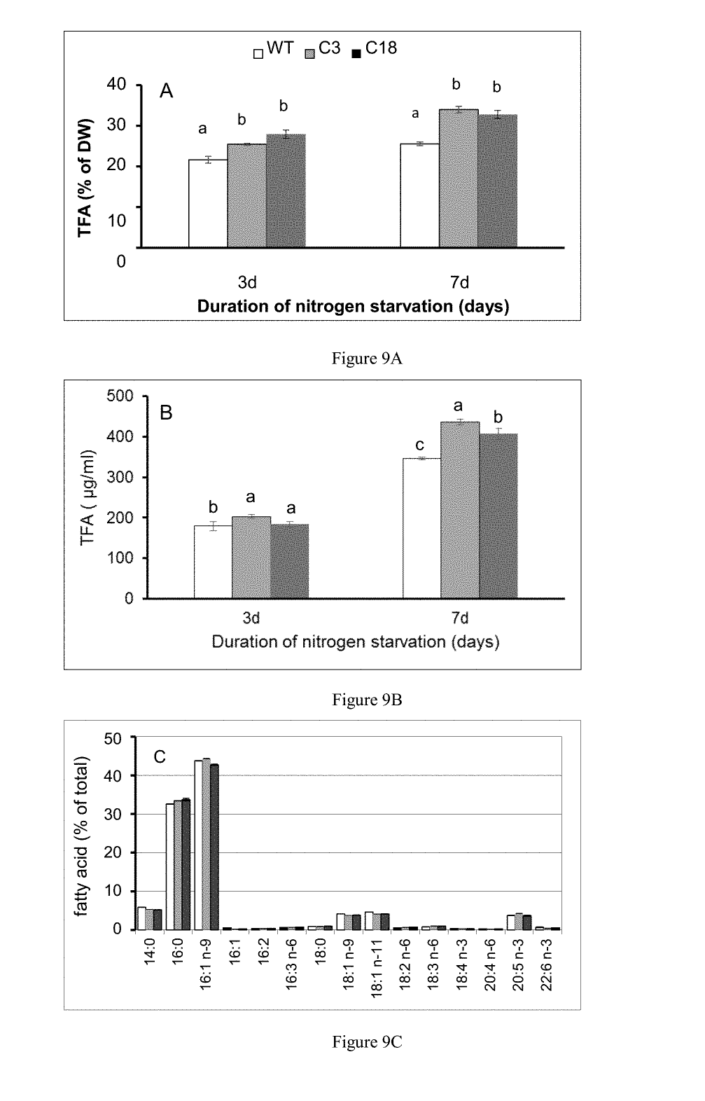

In order to monitor LDs formation and GFP-signal localization in the recombinant P. tricornutum cells, a simple and rapid approach to induce LDs formation in nutrient-sufficient cells, lacking LDs, by supplementation with free palmitoleic acid (16:1) was developed. Administration of 16:1 which is a major acyl component of TAG in P. tricornutum (along with palmitate, 16:0), led to rapid esterification of exogenous fatty acids into TAG and sequestration into LDs (FIG. 5). The uptake of the exogenous fatty acid was evident from the GC analysis of neutral lipids, it constituted for more than 90% of total fatty acids in the TAG and FFA pool after the treatment (not shown). TAG formed concurrently with a decrease in free fatty acid, whereas after washing and resuspension in a fresh nutrient medium, TAG was depleted within the next 24 hours along with the disappearance of LDs. This approach enabled fast and robust detection of GFP-HOGP on LDs driven by both promoters (FIG. 5B). Selected colonies were screened by confocal microscopy for GFP fluorescence 16-24 hours after addition of the free 16:1 to the growth medium of the engineered cells. On the other hand, no signal of 2 could be detected under nitrogen replete conditions in the GFP-HOGP transformed but non-supplemented cells (FIG. 5B). Notably, 24 hours following 16:1 administration, lines transformed with both constructs tested as positive for GFP, featured distinctive decoration of LDs with GFP fluorescence. It should be noted that such treatment did not impose severe stress to the cells. Though cell division stopped, growth recovered after removing of exogenous 16:1 by washing and resuspension in a fresh medium (FIGS. 5 and 9).

These results demonstrated that (i) HOGP is an LDs-localized protein, and (ii) that N-terminal fusion of GFP to this protein does not interfere with its correct intracellular localization on diatom LDs. However, only the GFP-HOGP constructs proved efficient, repeated attempts to express HOGP-GFP constructs (2 fused to C-terminus of HOGP) failed; this implying the importance of the C terminus of HOGP for the correct localization on LDs.

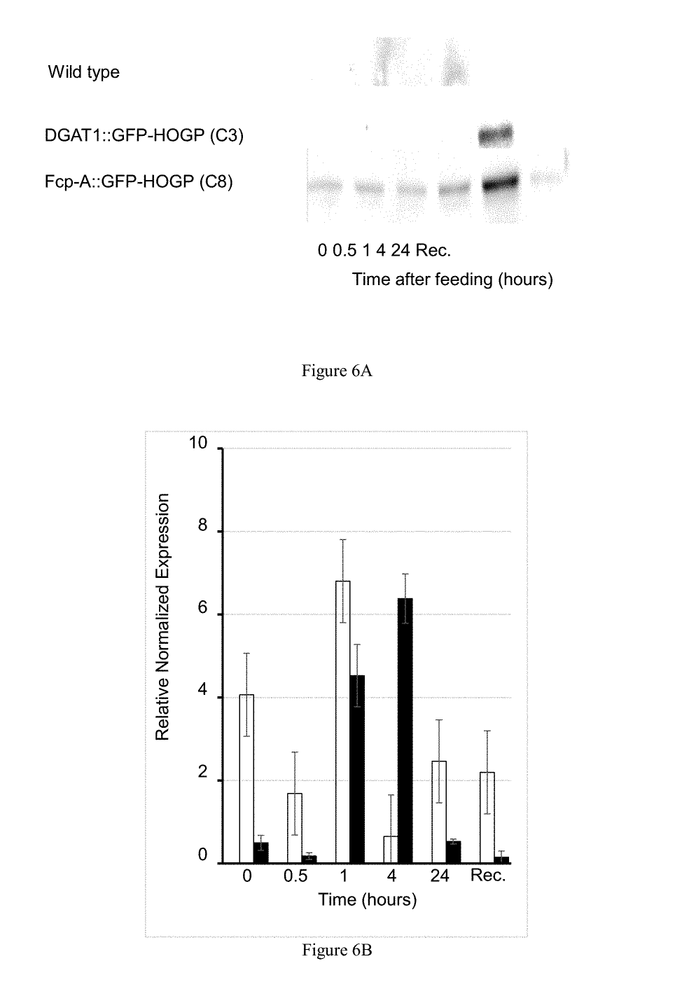

The results observed by fluorescence microscopy were essentially confirmed by the Western blot analysis of the amount of GFP-HOGP protein in the cells using anti-GFP antibodies (FIG. 6A). As expected, no GFP signal was detected in the wild type, whereas strain T1:EGFP-HOGP constantly expressed a low but detectable level of EGFP-HOGP; after 24 hours of FFA administration a much higher level of EGFP-HOGP could be detected. In strain transformed with DGAT1:EGFP-HOGP, immunoblotting detection was achieved only after 24 h from induction of LDs formation by FFA feeding.

The above described results revealed that the cloned fragment of the P. tricornutum DGAT1 promoter is efficient in expressing genes under conditions associated with intensive acyl groups esterification and deposition to LDs. Using HOGP-specific primers, EGFP-HOGP expression patterns were further investigated by qPCR under the control of two different promoters, DGAT1 and fcpA, in the course of palmitoleic acid feeding (FIG. 6B). The extent and the pattern of the transcriptional activation of HOGP under the fcpA promoter was intrinsically different from that under the DGAT1 promoter. Under control of the fcpA promoter, EGFP-HOGP expression at time zero was relatively high, and reached its highest level after one hour post-feeding, following a sharp drop after 4 hrs. Under the control of the DGAT1 promoter, at time zero transcript levels for HOGP were very low; one hour after induction of LDs formation by FFA feeding, the transcript levels rose, and remained elevated after four hours as well. Under the control of both promoters, 24 hours after induction, the transcript levels returned to those of time zero. These data confirmed the transient activation of target gene expression, triggered by the FFA feeding.

Expression of GFP-HOGP Driven by the Inducible DGAT1 Promoter Directs GFP to Lipid Droplets Under Nitrogen-Starvation

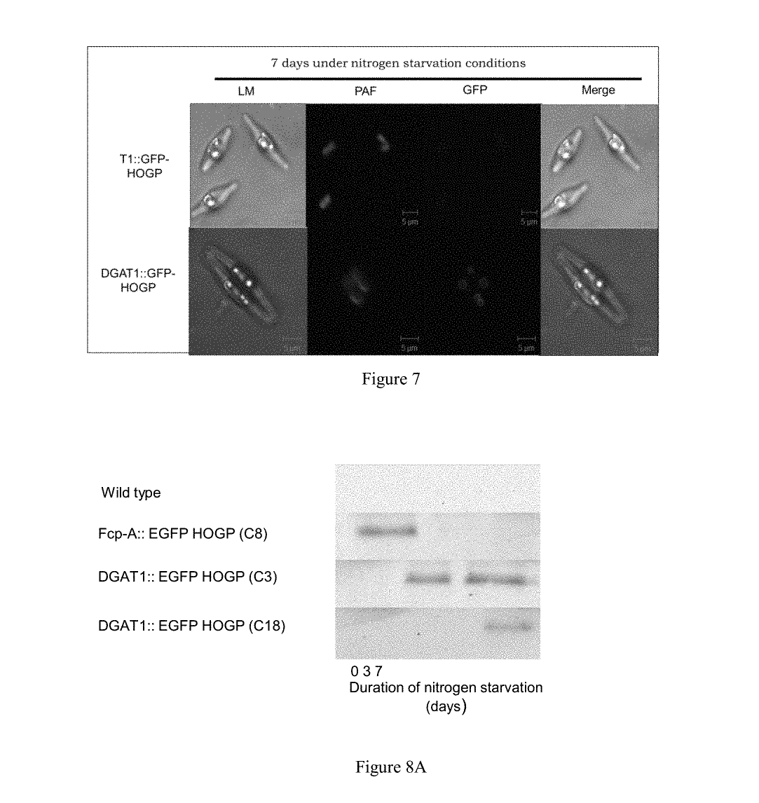

Using a FFA-feeding approach, it was possible to establish assay conditions under which LDs were formed and the fcpA promoter was active, hence it was possible to monitor LDs formation under nitrogen-replete conditions, ensuring that present target protein HOGP is indeed a LDs' protein. However, under conditions of nitrogen starvation which triggers TAG formation from endogenous acyl groups, recombinant lines obtained using the fcpA promoter construct did not reveal any GFP fluorescence. On the other hand, cells expressing the fusion protein under the control of the DGAT1 promoter, which is strongly induced under nitrogen starvation, revealed a bright green fluorescence signal localized to LDs (FIG. 7). Thus, GFP-HOGP under control of the fcpA promoter is insufficiently expressed under nitrogen starvation to be visualized on P. tricornutum LDs or in other cellular locations.

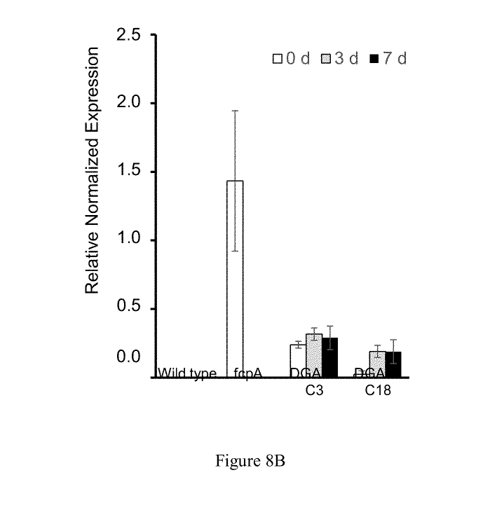

In order to assess regulation of the EGFP-HOGP expression in the course of nitrogen starvation, EGFP::HOGP expression was quantified in one fcpA::GFP-HOGP transformant and in two different DGAT1::EGFP-HOGP lines (C3 and C18) by immuno-staining of Western blots using anti-GFP antibody (FIG. 8A). The fcpA-transformant strain (C8) revealed a significant signal for the HOGP-GFP fusion protein before onset of the nitrogen starvation, but no signals were detected after 3 and 7 days of nitrogen starvation. In contrast, strain C18 (DGAT1::GFP-HOGP) revealed a signal only after seven days of nitrogen starvation; remarkably, strain C3 (DGAT1::GFP-HOGP) showed a strong signal for EGFP-HOGP already 3 days after onset of nitrogen starvation as well as GFP fluorescence (not shown).

No fluorescent protein is observed upon expression using the fcpA promoter; expression using the DGAT1 promoter (strain C3) results in GFP fluorescence localized on the surface of LDs. LM: light microscopy; PAF: photosynthetic apparatus fluorescence; GFP: GFP-HOGP fusion protein fluorescence; Merge: overlay of LM and GFP (FIG. 7).

In order to further clarify the expression mechanism of the fusion protein under the two promoters, HOGP-GFP fusion protein mRNA levels was quantified by qPCR using HOGP specific primers (Table 2). The result presented in FIG. 4B clearly shows that the fcpA promoter is very rapidly and very efficiently shut down under nitrogen starvation, with essentially no detectable mRNA remaining after 3 days of starvation. In contrast, mRNA levels in strain DGAT1-GFP-HOGP (C18) were near zero at nutrient replete conditions, but increased strongly both after 3 and 7 days of starvation. However, even at these time points, mRNA levels remain far below those of the fcpA promoter driven expression under nitrogen-replete conditions. Interestingly, in strain DGAT1-GFP-HOGP (C3), mRNA levels at time zero were already significant, and increased only slightly after three days and seven days of starvation. Nevertheless, the fusion protein becomes visible by fluorescent microscopy and immune-staining after three days of starvation, while no protein is detected at day zero, when no LDs were formed. The combined results of those analyses indicate that strong post-transcriptional mechanisms seem to be active in controlling fusion protein accumulation, besides mere transcriptional control. The strong difference between strains C3 and C18 indicates that the insertion location of the construct provides additional regulatory signals for gene expression, with strain C18 showing clearly regulated induction of mRNA accumulation, while such regulation is almost absent in strain C3.

Overexpression of HOGP Leads to Enhanced TFA and TAG Production and does not Impact Fatty Acid Composition of TAG Under Nitrogen Starvation

Total FA (TFA) and TAG contents were quantified (FIG. 9) in wild type and transformant strains C3 and C18 after 3 and 7 days of nitrogen starvation. After optimizing initial cell density and log-phase growth in an incubator shaker (see Methods), cells were transferred to nitrogen free medium. After 7 days of nitrogen starvation the total fatty acid content (TFA) of wild type accounted for about than 25% of the cell dry weight. Two transformant lines revealed both significantly higher TFA and TAG contents than the wild type. The transformant strains C3 and C18 had 25-40% percent increased total FA content (expressed both as TFA content out of the cell dry weight and volumetric culture concentration) than wild type after 3 days and 7 days of starvation (FIG. 9A). These strains also demonstrated elevated TAG content (% of DW) after 7 days of starvation (29.6±0.09 (C3); 29.4±0.39 (C18) as compared to 24.5±0.70 in the wild type, p<0.001). The results of the above described analyses, showed a significant effect of HOGP overexpression on TFA and TAG content in the biomass of transgenic lines compared to the wild type after 3 and 7 days upon transfer to nitrogen starvation. However, expression of GFP-HOGP did not alter the fatty acid composition of total lipids (not shown) as well as that of TAG (FIG. 9C), suggesting that present manipulations of GFP-HOGP expression did not affect either activity or acyl preference of TAG assembly enzymes. The major acyl groups accumulating in TAG under nitrogen starvation conditions were palmitate (above 30% of TFA) and palmitoleate (above 40% of TFA), whereas the proportions of the major LC-PUFA, EPA, amounted for less than 5% percent.

In spite of ever increasing interest in the biogenesis of LDs in microalgae, the basic molecular tools for the disclosure of this mechanism are still scarce. Herein, it was demonstrated the significance of overexpressing genes encoding LDs-associated proteins using suitable promoters. This is the first report, in P. tricornutum, of an endogenous promoter inducing gene expression under prolonged nitrogen starvation conditions applied successfully. Moreover, it is also the first report of successfully in vivo marking and localization of an LDs' protein in the diatom P. tricornutum, by GFP bound to a green alga LD protein. Exploring two assay conditions, this study thus provides irrefutable evidence to the assumption, that HOGP is a LDs-associated protein. Such evidence has been lacking in the previous studies on HOGP. On the one hand, the results indicated that LDs structures are sufficiently conserved between green algae and diatoms to allow for mutually exchangeable functional complementation. The present results imply that LDs proteins of evolutionary distinctive organisms can be successfully accommodated on LDs in heterologous hosts presumably owing to their hydropathy properties which allow anchoring of the major LDs protein of a green alga on the LD surface of a diatom. Likewise, the major LDSP of the microalga Nannochloropsis oceanica was expressed in an oleosin-deficient mutant of Arabidopsis (Vieler et al., 2012).

The PtDGAT1 gene transcript (the spliced form) expression patterns has been shown to be upregulated by more than threefold under nitrogen starvation, whereas unspliced transcript (due to intron retention) was induced at much higher level (Guihéneuf et al., 2011). Indeed, by using nitrogen-starvation inducible DGAT1 promoter, it was possible to locate GFP-HOGP fluorescence signal at the expected cell location—on the LDs—whose formation was induced by two means, by the exogenous free fatty acid feeding and by the onset of nitrogen starvation. The fcpA promoter, a promoter for a photosynthetic gene, appeared to be active only under nitrogen replete conditions or at the very early stages of nitrogen starvation. Using it as reference promoter, neither transcripts expression nor GFP-HOGP fluorescence signal under nitrogen starvation conditions, were detected.

Administration of free palmitoleic acid was proven to successfully induce LDs formation in P. tricornutum. The primary advantage of the developed assay is a rapid screening of transformant colonies expressing FP-fused LDs-associated proteins. Free fatty acid feeding is widely employed in the studies on LDs biogenesis in mammalian and yeast cells, but is still rarely explored in microalgae for the purposes of LDs formation monitoring. The palmitoleic acid (16:1), the major fatty acid component of TAG in P. tricornutum, which is well soluble in the nutrient medium when supplied as a DMSO solution was used. High contents of free fatty acids are toxic to the cells and, hence, should be isolated and sequestered within LDs in order to avoid their harmful effect on cell membranes and organelles. Specifically in P. tricornutum, feeding different, primarily omega-3 polyunsaturated fatty acids, and, in particular, its major LC-PUFA, EPA, caused photosynthetic pigments degradation, chloroplast membrane deterioration and stimulated release of volatile compounds implicated in cell defense in diatoms (Schobert and Elstner, 1980). In this earlier study, 16:1 exerted less stressful effect, did not promote intensive volatile chemical production and even decreased the release of hexanal. Based on these observations, it is possible now to explain these observations by an efficient capacity of P. tricornutum to sequester 16:1 in LDs.

From 25 positive clones expressing GFP-HOGP, two clones were chosen as representative ones, one clone that showing a strong fluorescence signal (C3), and one (C18) showing a moderate fluorescence signal. HOGP-expressing clones enhanced TFA production in engineered strains under nitrogen starvation by up to 25 percent as compared to the wild type. Under the optimized nitrogen starvation conditions for rapid induction of TAG biosynthesis in P. tricornutum, TAG content in wild type reached 25% of dry weight within 7 days. In transformant lines even higher values of up to 30% were achieved. The fact that overexpression of HOGP in P. tricornutum gave rise to significantly enhanced TAG (oil) productivity, furthermore argues for the biotechnological relevance of this protein in regulating LD biosynthesis and TAG accumulation. It must be further determined whether HOGP overexpression facilitates biosynthesis and accumulation of TAG, or interferes with TAG degradation by lipases. As reported by Trentacoste et al. (2013), down-regulation of lipase activity in the diatom Thalassiosira pseudonana resulted in enhanced TAG content under nutrient-replete conditions. It is thus possible that localization of HOGP on the surface of LDs premediates increased stability and storage capacity of LDs, for example by reducing accessibility to lipases that have been shown to reduce oil accumulation in diatoms (Trentacoste et al. 2013). It is expected that the effect achieved might be further complemented (increased, enhanced) by the additional impact of acyltransferase overexpression that may result in synergistically increased TAG productivity. Overexpression of acyltransferases, participating in the reactions of TAG assembly, has been shown to enhance TAG formation in microalgae when adequate gene promoters tuned for certain cultivation conditions were utilized (Iwai et al., 2014; Iskandarov et al., 2015). It can therefore be expected that the efforts presented here will assist in rapid advances both at the level of understanding basic algal cell biology related to LD biogenesis, and also in gaining further insights into the biotechnologically important question of enhancing oil production by microalgae.

### Example 3: Inducible Transgene Expression in Phaeodactylum Tricornutum Using Novel Endogenous Promoters Drives Enhanced TAG Accumulation by Overexpression of Endogenous Dicylglycerol Transferase DGAT1 or Foreign Oil Globule Protein HOGP

The vector pPHA-T1 was modified such that the fcp-A promoter was replaced by either the DGAT1 or ammonium transporter (AT) promoter as described previously (Table 3).

TABLE 3
plasmid constructs

| Promoter | Gene |
|---|---|
| Ammonium | Full-length PtDGAT1 |
| transporter | HOGP (AT::mKate2-HOGP) |
| DGAT1 | Full-length PtDGAT1 |

* AT—ammonium transporter

The constructs were: AT promoter directly coupled and/or connected to full length PtDGAT1; and DGAT1 promoter directly coupled and/or connected to full length PtDGAT1.

The full-length PtDGAT1 open reading frame [ORF] was obtained from cDNA isolated from P. tricornutum grown under nitrogen starvation; it is 2271 bp in length, coding for the corresponding protein of 756 amino acids (Guihéneuf et al. 2011b). The full-length PtDGAT1 gene was cloned into pJET 1.2 vector (Thermo Scientific, Lithuania)

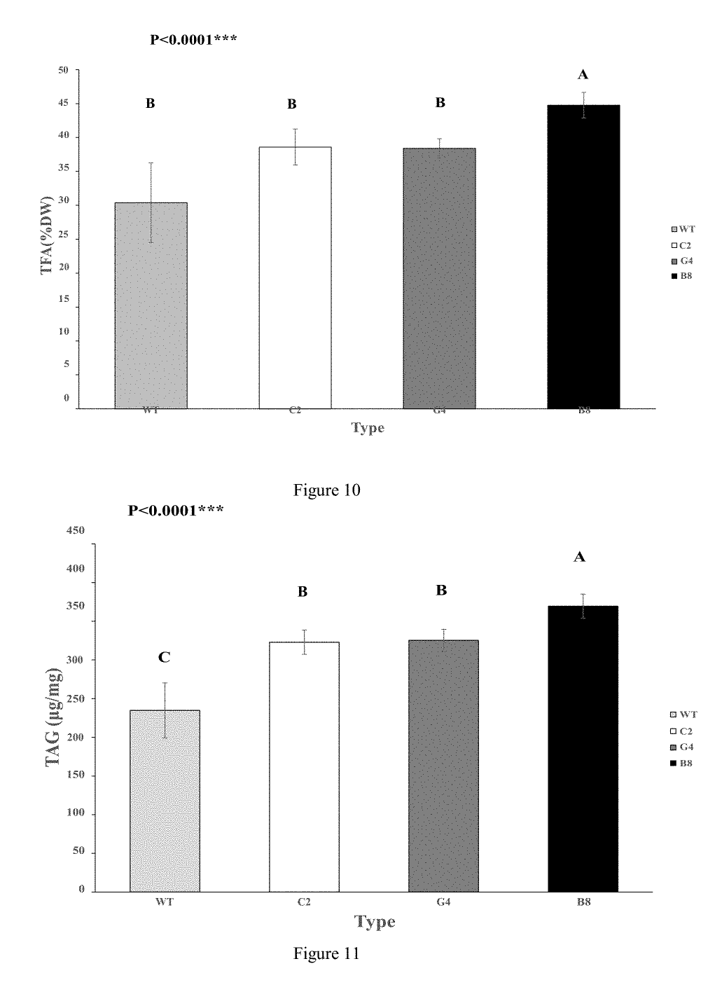

TFA and TAG content analyses, 3 and 7 days after induction of nitrogen starvation, show a significant effect of HOGP in the transgenic lines compare to the wild, which mean HOGP creating a shift from polar lipid toward TAG production. FIGS. 10 and 11 show fatty acid and TAG accumulation in wt and 3 different P. tricornutum transformants expressing mKATE2-HOGP fusion under the control of the strong, inducible AT promoter. Two transformant strains display about 25% increased TAG and FA content after 7 days of starvation, while strain B8 shows 50% increased lipid content. To what extend that is related to the higher gene expression level achieved by this promoter remains to be quantified.

Over-Expression of Full-Length PtDGAT1 Driven by DGAT1 and AT Promoter in P. tricornutum Results in the Enhancement of TAG Formation

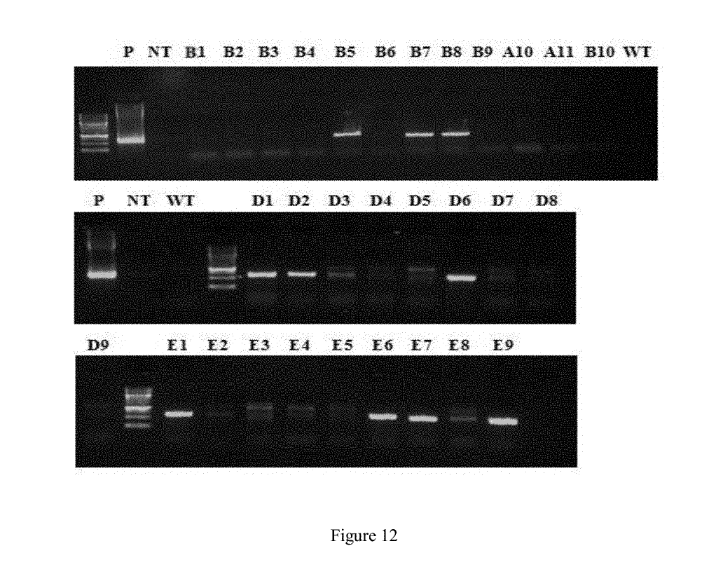

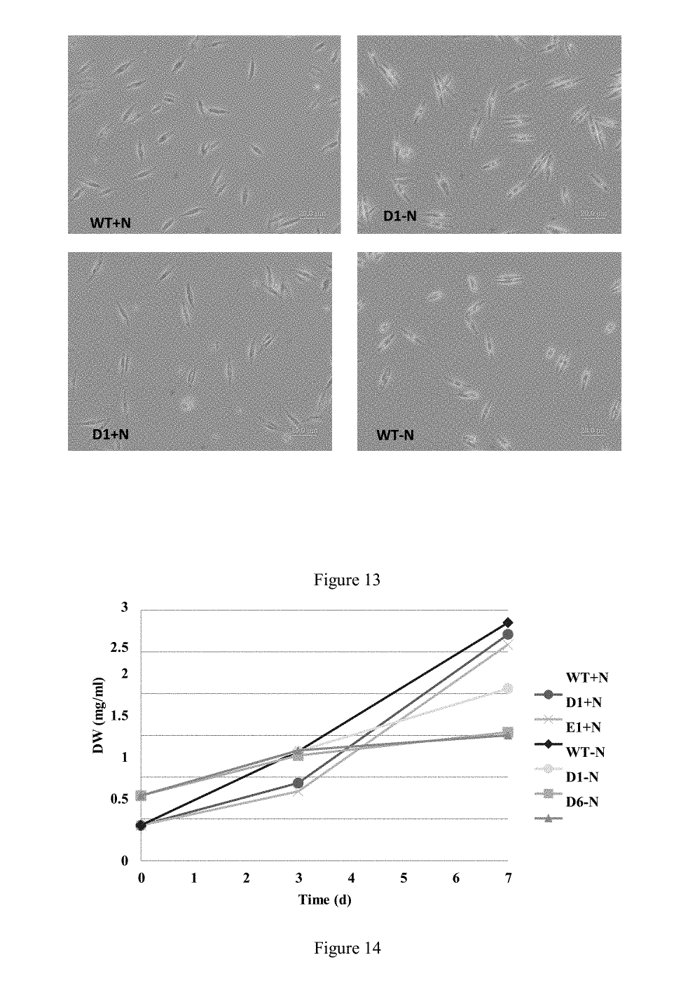

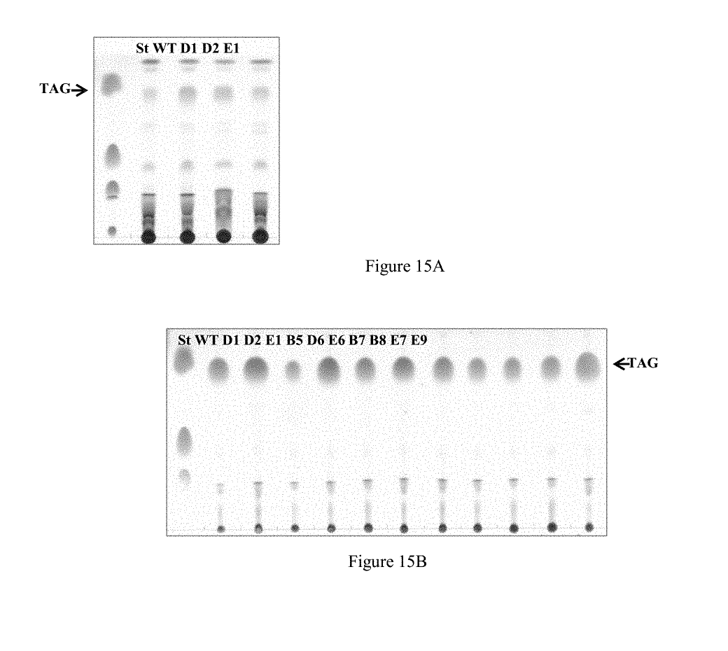

DGAT1 catalyzes the terminal step of TAG assembly and therefore its overexpression may increase TAG formation, as it was shown in several cases studied in plants, mammals and yeast. TAG accumulation in P. tricornutum after DGAT1-overexpression under the control of two different P. tricornitum promoters the ammonium-transporter (AT) gene promoter, and the PtDGAT1 promoter that is induced under nitrogen starvation, were compared. Transformed colonies that were resistant to antibiotic were screened by PCR for the presence of the cloned genes. Ten PCR-positive colonies (FIG. 12) were chosen for lipid analyses: AT promoter-full length DGAT1: D1, D2, E1, DGAT1 promoter:: DGAT1: D6, E6, B5, E7, E9, B7, B8. After 3 and 7 days of growth, cultures were sampled for TAG content and FA profile analysis. The growth of the WT and transformants was estimated by measuring the DW (Error! Reference source not found.). In the full medium he growth of transformants (possessing AT promoter: PtDGAT1) was similar to the WT. On the other hand, under nitrogen starvation D1 (possessing AT promoter: PtDGAT1) and D6 (possessing DGAT1 promoter: PtDGAT1) reached lower DW than the WT after 7 days.

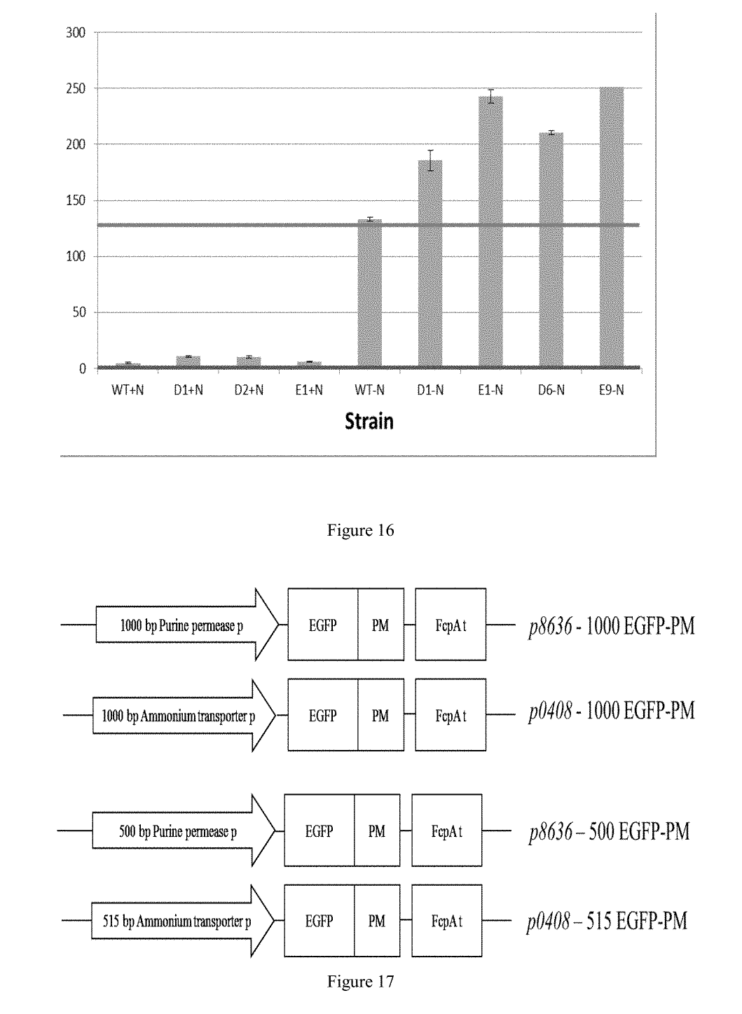

TAG content is presented as μg of TFA measured in the TAG normalized to unit of DW (mg) of dried cells after 3 and 7 days respectively. In full nitrogen replete medium after 3 days, the lines D1 and D2 that were transformed with the construct AT promoter:: PtDGAT1 accumulated 7.4 and 4.1 times more TAG than the WT, respectively. Yet, these values were much lower than the TAG accumulated in the nitrogen-deficient cultures. Under nitrogen starvation, significant differences between the lines were observed only at day 7: (1) the lines D1 and E1 that contain AT promoter:: PtDGAT1 accumulated 1.4 and 1.8 times more TAG than the WT respectively; (2) the line D6 that was obtained following the transformation with the vector DGAT1 promoter::PtDGAT1 accumulated 1.6 times more TAG than the WT; highest TAG accumulations were observed in strains E1 and E9 after seven days of nitrogen starvation (FIG. 16).

1. A nucleic acid molecule comprising DGAT1 promoter or an active fragment thereof coupled to a gene, wherein said gene is not controlled endogenically by the DGAT1 promoter.

2. A nucleic acid molecule comprising ammonium transporter promoter or an active fragment thereof coupled to a gene, wherein said gene is not controlled endogenically by the ammonium transporter promoter.

3. A nucleic acid molecule comprising putative purine permease promoter or an active fragment thereof coupled to a gene, wherein said gene is not controlled endogenically by the putative purine permease promoter.

4. The nucleic acid molecule of claim 1, wherein said DGAT1 promoter comprises SEQ ID NO: 1.

5. The nucleic acid molecule of claim 2, wherein said ammonium transporter promoter comprises SEQ ID NO: 2, SEQ ID NO: 30, SEQ ID NO: 32 or any combination thereof.

6. The nucleic acid molecule of claim 3, wherein said putative purine permease promoter comprises SEQ ID NO: 3, SEQ ID NO: 29, SEQ ID NO: 31, or any combination thereof.

7. (canceled)

8. The nucleic acid molecule of claim 1, wherein said gene is a fatty acid biosynthesis enzyme.

9-11. (canceled)

12. A cell transformed by the nucleic acid molecule of claim 1.

13. A method for enhancing the production of a protein, comprising the steps of: (a) preparing a composite nucleic acid molecule of claim 1; and (b) transforming a cell with the nucleic acid molecule, thereby enhancing the production of a protein in a cell.

14. (canceled)

15. A composition comprising the protein of claim 13 and an acceptable carrier.

16. A composition comprising the protein of claim 15 and an acceptable carrier.

17. A composition comprising the protein of claim 16 and an acceptable carrier.

18. The nucleic acid molecule of claim 2, wherein said gene is a fatty acid biosynthesis enzyme.

19. The nucleic acid molecule of claim 3, wherein said gene is a fatty acid biosynthesis enzyme.

20. A cell transformed by the nucleic acid molecule of claim 2.

21. A cell transformed by the nucleic acid molecule of claim 3.

22. A method for enhancing the production of a protein, comprising the steps of: (a) preparing a composite nucleic acid molecule of claim 2; and (b) transforming a cell with the nucleic acid molecule, thereby enhancing the production of a protein in a cell.

23. method for enhancing the production of a protein, comprising the steps of: (a) preparing a composite nucleic acid molecule of claim 3; and (b) transforming a cell with the nucleic acid molecule, thereby enhancing the production of a protein in a cell.


SEQUENCE LISTING (32 sequences)

SEQ ID NO: 1 - DNA, 2150 bp, Phaeodactylum tricornutum
atcagcgtctcgcctccacctccgttttgttcccgtacgtcattgattaaatccgtatcaacaccgagggagccatccccatcacgatagagaatattcacaaactgatagccaatatttatatccacaaagtcgtaaatcttttgagcgtaaacatccgacgctgagacgttcgaaacaccgttcaatgagactaaaacttggttgaattgaagagattctcgaacatcgtcacagttgttgagacgcgatggccaggaaccaccgtgtcgacgaatctgtcgacgcagtcggggtttcaataggggggaatgctcgtcaaggacgtggcgtgccaaccagactcgcttaaaaaacgggtggtccgaggcttcgatcaccatcttggaaaatatctgcttactaacaagccttgccgacgggtcttcatccaatgcttgctgatcacggcgatgataacgacccaaaaaatttcgactgataacagatgtcaacaaatcgaatctcgtcgattctgctatgctacttgtgtcactcgcagtttcacctgaaatgctttgggcggccgaattccggtcattttcaaaagactcctgtaaggagggatcggcatcactcgcatcgacattggcaacgacattcagggttgcgtccatgatttcgccaccaacttcattgtagagtcgattgatgattcgaaattccaacaccggacagggctctttctcttcgtcgtccgaagattcgtcgtgcccgtgattgggatcaaaatcgttgtttcgccgtaccccaggaccaaaccgaataacaatcggatcactaaagatcacttgtgcgtgtgactgaatacgcaatactttgccaaacaaaattgcgccacaaaagccagagtataacacccccaacaaagcttcgagcgagcatataaaattgataaagaaacaatgggttgggctcgagttttggtagctcaaagctggcgccgtcgaaccatatccgacggtcgaaaaagttgtccacgacaaggcgaaggcatcagcaaaggctgcgtctgtggacgcaaagagctctccgccaattcgtacacattcctcatcaattctaccggccatggttatgaaccccgcaaatagaatcacgagagcaaaaaacattacacacatgaccacgaacaagaatagaaaattgacacggaacatccaatgcaaatacatcgcaagtttttggttggggtttatgccaatggcgagaccagtcgaacggcggggtgagccaattcttccgttgcacgtcgtgattttccaaaacagagactgttgagggcttcgtcgggtggcttctagacgatactgcttcgtttgttgatgttggttcgatgcttccacgtcctccgactgttcttcgctcttagaaaaaacgtttaaaaatttggttttcagacgttgcccccatggcatggcggtgccttcatccatacgccgaagctacatgtcacaaagaagaatcagcaagtagaatgagtaactgaaaaaggaacatttccgcggccgttcgacttgttcaacttaacctacatgaacacattgcgctcgagatatttggtttataatgggctgaccgccttttatatttgacgtcatagttcccggctcgatcttcgggtttcccacgaaggaatcgtagaatacaaccgcgaacgatgtctatgcggagcaaaaggaaggagtagcctaaggttgtaaaggggacagaaacagcaaaacgttggttcacagtcaaacgtttcatgccgggtcgcggttcattttggtgtgtgttttggaatgcgcgaggttgcaaactgtgtacgtttataatttaaataccttttcaaattatagatattttgacatttcctaacgttagtaagtattgctccggtctgcttccaactgtaaactcaccctgacagtgagtgagtctctattttcttcgcattttggagaactcaggccggacaaaatcgttcacttcttgtataaacgggcatctttatcttaccgctgcattttccttcgtacaaagagttaagagtccaattctaatttttttcttgccagacagctagtcccaacaaggagacaggaggggctacacataccacagacacg

SEQ ID NO: 2 - DNA, 2003 bp, Phaeodactylum tricornutum
gggacccaacaaaaccaccatcgatccgattgcaatggcgactagaggagggacggcagccgaaggcacagagtttttcttgttggtcagcatgaaaatacaaaagaccaaaaaagcagtgccaaacgcttccaacaagaaggcgtgccacccacttgtcacatacttcgaaaggctgcggtgcgtaaaaaggaaaaaatggacgtcagcttgtgttttgaggagcaaggttttggtgaaaggcgcttgcagatccgttgagaagatattacctccaatagtcgccaaaagcagctgccgattgaattgaggcttgtccccctcgaacaattccgttcttcgcctcatagcttaaaatggcttcgtgataaatgattagatttgtcaaggcgccgagaaaagctcctgccagctgagcagcccagtagggaaggacgttagcgtagggaaaatcagccggacgaacgacagcgaacgataaagttacggctggattcaagtgtccaccggacaaggcagcggacgaataaagacccaaggtggctcccaggatccagacaatcgctgcctgccacatgccatccaagacaccgagatagattgctaggcaattggcgccacaaccgacttgcactagaatcgccgtcccaataagttccgccgtcaactgagagcgaagcgggggaggaggagcatcggtaatttgtggttctgtttgcggtatggatcctgaaaaagaatgacgattgaagcaatttgtatatcagccaggcaatacttcttggtcgcttgtaaagcatttgctcctcgattccaaagacggaacgcgaactcaccgtactcaaccatcttgtcttctttgttgcaaaaacagcttcagaaatttttggctctggataagtttacattcggtgcgtgtacgatcgaacttttacagtaagaacgtcgtccatcctggagaatcgagatcgatgttgactggaaagatccaggaataattgtcgagattcgcgctgtgcgtgacacagatgacacttcgcaaaacattttggcaaaagaagttcgtaaaagttgacttcagtgacataagggttactgtgtttagggttcgtcaaattttggcaccagagatgacgcatggagctagagaaagatcgaagccgtctgaagtgagaatgctcttctcgaagttatgaatagattagtgattagccgatattccggcaatcccttcactgtgaggcattcaatttaatttatctcactgtttggatatcctttgttgtgaagcattccttttgtatggctttaggtggaagaaaatgctctcctttgatgctgccgcttgtcgatatcagccctcacccccaagtcgtcaagccaagatgagtaacagtgaggatttgtactttccaaaggcttgatcactggctcatccaaaacgattttttgtagtggaatcctacgtctcgtactgttgataagaaaagctatttatggtctatggttgaacgacgtaagaacggtcctgtgagaattctgtaaaacgacggagacacaaattcaagtggtactctctaattatgctggtataggccttgaaatgactgacgcgccactgttctccgacgagccaatcaaaacctttcaatcttacctggccgttgccttctctcgaacaattaatctggagttctgaattcgatttaatattttctgtctttatcgacgcaatcttattggatgactaagcttcgataggattgtagtggctggaaatgtcgtttgattgtgcaagcgtgtcaattgcgataacagagttgatgattggcccagggcaattatataggatcaaccgtcctgtgaagttggtaatgattctacataccggaaactgctacgctgtgcttctactaaaccttccctcacacctccttgatcagatcggaatttccgtttgcaccctggcggatccgacctccgtaactttcataaaactctgt

SEQ ID NO: 3 - DNA, 1998 bp, Phaeodactylum tricornutum
tactgcagaccgtgccgtagccaaattcgtcgttctggatgaagatgggcgccaaaagtcgttccagctctatccccacgttcgtatccggcaagtgctcgtctctcggcatttgggtatcgtgcaacagatttaacaggtcgtcgtgattccaagtttccttggcttctaaacgtactgttttctccaacaaagatttcagacgctctgttttaggccagggcgtgtccaacaatccattgcaaagcccgtacaccctccccggttctagaacgtgcacttgttgcggatcactttttgggtgtcggttggacacgtgacacaattgattgccatcgtacgcgagtaaattgaagccgttgtaatcctcaccatgcgaatgtacgcttcgacaatactcggcagcggacagttcgacgtcggttcggtcgcgttgacaaaagccgacgaccagatcgccgcgagagtgcttgtcttcgaattccttcggaccgctgcggtgattcgtcacgacagcaactcgggatgaaccaacgcgcgctgctagccagctaccacaagactgatcgtccaagccagcgacgagcgctccatcggaccaaacgtgcgctggtctcgagggtcgtgcgtagaactcgtcacgattcgtaacgagaatcaaggcgtacgaggggtgaatgttgacaccacaagcgacgatgcacatgatggcgtgcagaatgcgtaatgccgccagtttcaagaaagttaggtaatacttatcgattgattgatttactgttgattcggtttgtgagtttgtgaggttgctatccatcaccgtttggagtcgatggagcgacagtgaagtccacggttcgtatccgctcttgttaccctgtacgacttttgatgtgtgatcacgtgatgattctcattcttgaaactgatgtaacatcaattgtatcgactgacagtcacaggcagtcgatctggatgtcgtttggacacaacgaaagcacgtggtattgtcaactccgggtctggggccgcttctccggtgtctactcgtgcggtgtatgcttcggtacagtgctacaacggatgacagacgtgcggcaatagttcgttgtccaagtcgtttcgagccttgacatactttataccagagtgccaacttacaattctgtgctatgtttcgatgaggttcttcggacaaaaaaatcatggtaacagatgctgcttctcttgtcagtatctaacgaaattgactaactgtgagaatattacggagataaacggcgtccccgtacgtggcgaaatggacaaatgggaatcggaattgacggtgggtaggcggctatttatgaaagaaaaccggaacgacagatggagttgggacaaatcaaaactaaaatagtaccatcgatagccaatcatttcgaggtgtgtcgattccgcattcgtaagttctgaagtttttcggggaagctgagccagcggtcgattaccgttaaaatagtatattttgcgacaacttcgcttttctctatttctgagctagttatgctttacttgatatcattcgtttttcatcgttaagtgatgagttcgtgaacgctgcagcgttttgatcaacttcccttatcagattgggaggttgtgttctagagtgggattcccagcagtagaagtgtcgttgattggcttggttgaagcccggtacagcaatgattgggaggtgccagtgtcaaagttttccgccactccagcccgatatctttcttctctccactacggaatgaagaacggattttctgctactttttcttttcccatctgccataaattttcgttctgcataatttcatcgcagtgcaacgatattttatacacgttactaattgtttgtctgctttctcaacggttggcaattcgtcgtgtaaagtactcaagactattcaatccatttacgtgtgcaagagttgcaacaagaattccgctcttgtgtgaagcatc

SEQ ID NO: 4 - protein, 25 aa, Phaeodactylum tricornutum
SGLRSKLNPPDESGPGCMSCKCVLS

SEQ ID NO: 5 - DNA, 43 bp, Phaeodactylum tricornutum
aattcgatcgtcaggcctatggtgagcaagggcgaggagctgt

SEQ ID NO: 6 - DNA, 38 bp, Phaeodactylum tricornutum
agatctgagtccggacttgtacagctcgtccatgccga

SEQ ID NO: 7 - DNA, 94 bp, Phaeodactylum tricornutum
tccggactcagatctaagctgaaccctcctgatgagagtggccccggctgcatgagctgcaagtgtgtgctctcctgatctagagtcgacctgc

SEQ ID NO: 8 - DNA, 94 bp, Phaeodactylum tricornutum
gcaggtcgactctagatcaggagagcacacacttgcagctcatgcagccggggccactctcatcaggagggttcagcttagatctgagtccgga

SEQ ID NO: 9 - DNA, 39 bp, Phaeodactylum tricornutum
tgagagtgcaccatatgtactgcagaccgtgccgtagcc

SEQ ID NO: 10 - DNA, 40 bp, Phaeodactylum tricornutum
cttgctcaccataggcctgatgcttcacacaagagcggaa

SEQ ID NO: 11 - DNA, 35 bp, Phaeodactylum tricornutum
tgagagtgcaccatatggggacccaacaaaaccac

SEQ ID NO: 12 - DNA, 40 bp, Phaeodactylum tricornutum
cttgctcaccataggcctacagagttttatgaaagttacg

SEQ ID NO: 13 - DNA, 37 bp, Phaeodactylum tricornutum
tgagagtgcaccatatgtgacagatagctttcaaaag

SEQ ID NO: 14 - DNA, 37 bp, Phaeodactylum tricornutum
cttgctcaccataggcctgatgggatagagaggtgtt

SEQ ID NO: 15 - DNA, 42 bp, Phaeodactylum tricornutum
tgagagtgcaccatatgatcagcgtctcgcctccacctccgt

SEQ ID NO: 16 - DNA, 37 bp, Phaeodactylum tricornutum
cttgctcaccataggcctcgtgtctgtggtatgtgta

SEQ ID NO: 17 - DNA, 24 bp, Phaeodactylum tricornutum
cgtggatgatgaggactacgaatc

SEQ ID NO: 18 - DNA, 24 bp, Phaeodactylum tricornutum
ccacttcttattgcgacggtaaac

SEQ ID NO: 19 - DNA, 20 bp, Phaeodactylum tricornutum
aaggacgacggcaactacaa

SEQ ID NO: 20 - DNA, 23 bp, Phaeodactylum tricornutum
tctgcttgtcggccatgatatag

SEQ ID NO: 21 - DNA, 36 bp, Phaeodactylum tricornutum
tgagagtgcaccatatgcacgtggtattgtcaactc

SEQ ID NO: 22 - DNA, 37 bp, Phaeodactylum tricornutum
cttgctcaccataggcctgatgcttcacacaagagcg

SEQ ID NO: 23 - DNA, 35 bp, Phaeodactylum tricornutum
tgagagtgcaccatatggagattcgcgctgtgcgt

SEQ ID NO: 24 - DNA, 40 bp, Phaeodactylum tricornutum; identical to SEQ ID NO: 12

SEQ ID NO: 25 - DNA, 39 bp, Phaeodactylum tricornutum
tgagagtgcaccatatgtagtatattttgcgacaacttc

SEQ ID NO: 26 - DNA, 37 bp, Phaeodactylum tricornutum; identical to SEQ ID NO: 22

SEQ ID NO: 27 - DNA, 38 bp, Phaeodactylum tricornutum
tgagagtgcaccatatgagctatttatggtctatggtt

SEQ ID NO: 28 - DNA, 40 bp, Phaeodactylum tricornutum; identical to SEQ ID NO: 12

SEQ ID NO: 29 - DNA, 1000 bp, Phaeodactylum tricornutum
cacgtggtattgtcaactccgggtctggggccgcttctccggtgtctactcgtgcggtgtatgcttcggtacagtgctacaacggatgacagacgtgcggcaatagttcgttgtccaagtcgtttcgagccttgacatactttataccagagtgccaacttacaattctgtgctatgtttcgatgaggttcttcggacaaaaaaatcatggtaacagatgctgcttctcttgtcagtatctaacgaaattgactaactgtgagaatattacggagataaacggcgtccccgtacgtggcgaaatggacaaatgggaatcggaattgacggtgggtaggcggctatttatgaaagaaaaccggaacgacagatggagttgggacaaatcaaaactaaaatagtaccatcgatagccaatcatttcgaggtgtgtcgattccgcattcgtaagttctgaagtttttcggggaagctgagccagcggtcgattaccgttaaaatagtatattttgcgacaacttcgcttttctctatttctgagctagttatgctttacttgatatcattcgtttttcatcgttaagtgatgagttcgtgaacgctgcagcgttttgatcaacttcccttatcagattgggaggttgtgttctagagtgggattcccagcagtagaagtgtcgttgattggcttggttgaagcccggtacagcaatgattgggaggtgccagtgtcaaagttttccgccactccagcccgatatctttcttctctccactacggaatgaagaacggattttctgctactttttcttttcccatctgccataaattttcgttctgcataatttcatcgcagtgcaacgatattttatacacgttactaattgtttgtctgctttctcaacggttggcaattcgtcgtgtaaagtactcaagactattcaatccatttacgtgtgcaagagttgcaacaagaattccgctcttgtgtgaagcatc

SEQ ID NO: 30 - DNA, 1000 bp, Phaeodactylum tricornutum
gagattcgcgctgtgcgtgacacagatgacacttcgcaaaacattttggcaaaagaagttcgtaaaagttgacttcagtgacataagggttactgtgtttagggttcgtcaaattttggcaccagagatgacgcatggagctagagaaagatcgaagccgtctgaagtgagaatgctcttctcgaagttatgaatagattagtgattagccgatattccggcaatcccttcactgtgaggcattcaatttaatttatctcactgtttggatatcctttgttgtgaagcattccttttgtatggctttaggtggaagaaaatgctctcctttgatgctgccgcttgtcgatatcagccctcacccccaagtcgtcaagccaagatgagtaacagtgaggatttgtactttccaaaggcttgatcactggctcatccaaaacgattttttgtagtggaatcctacgtctcgtactgttgataagaaaagctatttatggtctatggttgaacgacgtaagaacggtcctgtgagaattctgtaaaacgacggagacacaaattcaagtggtactctctaattatgctggtataggccttgaaatgactgacgcgccactgttctccgacgagccaatcaaaacctttcaatcttacctggccgttgccttctctcgaacaattaatctggagttctgaattcgatttaatattttctgtctttatcgacgcaatcttattggatgactaagcttcgataggattgtagtggctggaaatgtcgtttgattgtgcaagcgtgtcaattgcgataacagagttgatgattggcccagggcaattatataggatcaaccgtcctgtgaagttggtaatgattctacataccggaaactgctacgctgtgcttctactaaaccttccctcacacctccttgatcagatcggaatttccgtttgcaccctggcggatccgacctccgtaactttcataaaactctgt

SEQ ID NO: 31 - DNA, 500 bp, Phaeodactylum tricornutum
tagtatattttgcgacaacttcgcttttctctatttctgagctagttatgctttacttgatatcattcgtttttcatcgttaagtgatgagttcgtgaacgctgcagcgttttgatcaacttcccttatcagattgggaggttgtgttctagagtgggattcccagcagtagaagtgtcgttgattggcttggttgaagcccggtacagcaatgattgggaggtgccagtgtcaaagttttccgccactccagcccgatatctttcttctctccactacggaatgaagaacggattttctgctactttttcttttcccatctgccataaattttcgttctgcataatttcatcgcagtgcaacgatattttatacacgttactaattgtttgtctgctttctcaacggttggcaattcgtcgtgtaaagtactcaagactattcaatccatttacgtgtgcaagagttgcaacaagaattccgctcttgtgtgaagcatc

SEQ ID NO: 32 - DNA, 515 bp, Phaeodactylum tricornutum
agctatttatggtctatggttgaacgacgtaagaacggtcctgtgagaattctgtaaaacgacggagacacaaattcaagtggtactctctaattatgctggtataggccttgaaatgactgacgcgccactgttctccgacgagccaatcaaaacctttcaatcttacctggccgttgccttctctcgaacaattaatctggagttctgaattcgatttaatattttctgtctttatcgacgcaatcttattggatgactaagcttcgataggattgtagtggctggaaatgtcgtttgattgtgcaagcgtgtcaattgcgataacagagttgatgattggcccagggcaattatataggatcaaccgtcctgtgaagttggtaatgattctacataccggaaactgctacgctgtgcttctactaaaccttccctcacacctccttgatcagatcggaatttccgtttgcaccctggcggatccgacctccgtaactttcataaaactctgt